(19) United States
(12) Patent Application Publication
(10) Pub. No.: US 2021/0217518 A1
(43) Pub. Date: Jul. 15, 2021

(54) SYSTEM AND METHOD FOR PROVIDING FLUIDIC PERFORMANCE CHARACTERISTICS TO A DIAGNOSTIC PLATFORM

(71) Applicant: DIBA INDUSTRIES, INC., Danbury, CT (US)

(72) Inventor: Sean McKirdy, Newington, CT (US)

(21) Appl. No.: 16/967,564

(22) PCT Filed: Feb. 6, 2019
(86) PCT No.: PCT/US19/16902

§ 371 Date: Aug. 5, 2020

Related U.S. Application Data
(60) Provisional application No. 62/626,918, filed on Feb. 6, 2018.

(51) Int. Cl. G16H 40/67; G16H 40/20; G06K 19/07; G06K 7/10; H04W 4/80

(52) U.S. Cl. CPC G16H 40/67; G16H 40/20; H04W 4/80; G06K 19/0723; G06K 7/10297; G06K 19/0717

(57) ABSTRACT

A fluid monitoring system and method is provided and includes an RFID sensing device configured for wireless communication, a fluid reservoir, wherein the RFID sensing device is associated with the fluid reservoir, an interface device, wherein the interface device is configured to establish a communication link with the RFID sensing device and a remote processing device, and wherein the interface device includes, an RFID processing device configured to process RFID sensor data received by the RFID sensing device to generate a processed RFID result and a remote processing system, wherein the remote processing system is configured to, receive the processed RFID result from the RFID processing device, process the processed RFID result to generate output data, and operate responsive to the output data.

### RELATED APPLICATIONS

This application, which is the 371 US National Stage application of PCT Application Serial Number PCT/US19/16902, filed Feb. 6, 2019, is related to U.S. Provisional Patent Application Ser. No. 62/626,918 filed Feb. 6, 2018 and claims the benefit of priority of the filing dates of PCT Application Ser. No. PCT/US19/16902, filed Feb. 6, 2019, and U.S. Provisional Patent Application Ser. No. 62/626,918 filed Feb. 6, 2018, the contents of both of which are incorporated by reference herein in their entirety.

### FIELD OF THE INVENTION

The present invention relates generally to sensing devices for fluidic devices and more particularly to a fluidic diagnostic system having flow sensors which can provide real-time fluid flow data to improve system performance.

### BACKGROUND OF THE INVENTION

Diagnostic devices and/or platforms that are designed to analyze biological samples, such as blood, tissue, urine, DNA, etc., are well known and highly relied upon in the global healthcare industry. Typically, these devices rely on various fluidic components and sub-assemblies which work together to deliver reagents and samples to detection modules that are part of the diagnostic system. These fluidic components and sub-assemblies are often vital to the operation of the diagnostic system and, thus the flow performance characteristics of these fluidic components and sub-assemblies can have a large impact on the operational performance of the system and system software. Accordingly, it is beneficial to be able to monitor and measure the flow performance characteristics of these fluidic components and sub-assemblies.

The design engineers that are involved in the design and development of these diagnostic systems are required to evaluate and take into consideration, the tolerances and fluid management capabilities of the fluidic sub-assemblies and components as part of the overall system design. For example, one device uses a bonded thermoplastic manifold assembly to consolidate fluid path connections while also providing for more consistent fluid flow characteristics, wherein the bonded manifold assembly is fabricated using a manufacturing process which will determine the flow characteristics of the manifold assembly. This type of design typically requires a diagnostic system design engineer to evaluate the minimum/maximum flow range characteristics of the manifold assembly so it can predict how the system software will perform. Additionally, other diagnostic systems incorporate the use of real time flow sensors which can monitor and output the fluid flow performance characteristics of the fluidic assembly device and can either be mounted to the fluidic assembly (such as a bonded manifold or a tubing assembly) or connected in series with the fluidic assembly device.

Unfortunately however, there are several disadvantages to the incorporation of this sensing capability into the fluidic assembly. One such disadvantage is that fluidic sensors must be incorporated into the assembly and these sensors take up valuable space which can limit the design options. Additionally, because each of these sensors can cost hundreds of dollars per sensor, the overall cost of the fluidic assembly can be cost prohibitive. Furthermore, because each of the sensors require power to operate, power leads must be provided to power the sensors and additional power is needed to operate the sensors, thereby increasing operational costs. Another disadvantage is that these sensors may also have limitations due to fluid compatibility relative to the wetted surfaces of the sensor module. Moreover, currently the serviceability of the diagnostic platform is limited with respect to how failure mode and component utilization data is communicated between the provider of the fluidic component/assembly and the OEM of the diagnostic platform because of the inefficient communication channels between the provider of the fluidic component/assembly and the OEM of the diagnostic platform.

### SUMMARY OF THE INVENTION

A fluid monitoring system is provided and includes an RFID sensing device, wherein the RFID sensing device is configured for wireless communication, a fluid reservoir, wherein the RFID sensing device is associated with the fluid reservoir, an interface device, wherein the interface device is configured to establish a communication link with the RFID sensing device and a remote processing device, and wherein the interface device includes, an RFID processing device configured to process RFID sensor data received by the RFID sensing device to generate a processed RFID result and a remote processing system, wherein the remote processing system is configured to, receive the processed RFID result from the RFID processing device, process the processed RFID result to generate output data, and operate responsive to the output data.

A method for monitoring a fluid reservoir is provided and include configuring an interface device having a processor to establish a first communication link with an RFID sensing device associated with a fluid reservoir, processing data received from the RFID sensing device via the interface device to generate output data, configuring the interface device to establish a second communication link with a remote processing system, receiving the output data from the interface device, processing the output data via the remote processing system to generate resultant data and operating the remote processing system responsive to the resultant data.

An interface device for monitoring at least one characteristic of a fluid reservoir is provided and includes circuitry configured to establish a wireless communication link with an RFID sensor, at least one processor configured to process data that is received by the RFID sensor and generate output data, circuitry configured to establish a communication link with at least one computer system and a display device associated with the interface device, wherein the display screen provides a user interface and access to the interface device functionality.

### BRIEF DESCRIPTION OF THE DRAWINGS

The foregoing and other features and advantages of the present invention should be more fully understood from the accompanying detailed description of illustrative embodiments taken in conjunction with the following Figures in which like elements are numbered alike in the several Figures:

FIG. 1A is a schematic block diagram illustrating the test system, in accordance with one embodiment of the invention.

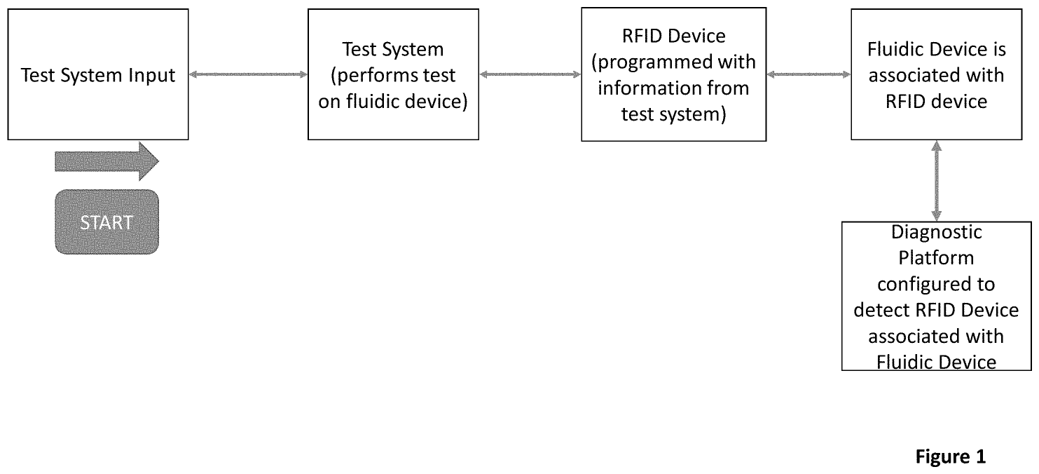

FIG. 1B is a schematic block diagram illustrating one portion of the test system of FIG. 1A.

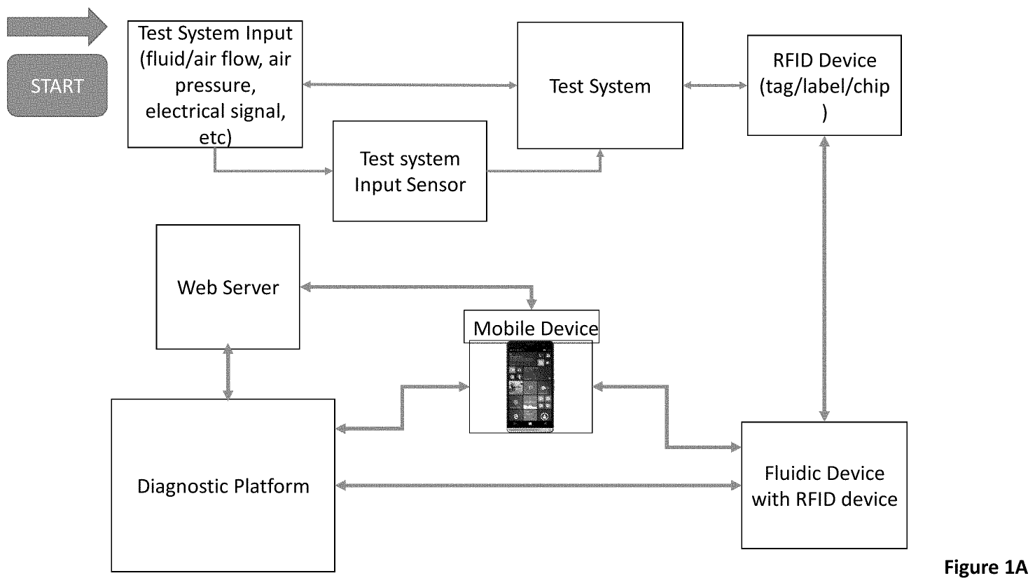

FIG. 1C are operational block diagrams for programming process and information viewing process for the test system of FIG. 1A.

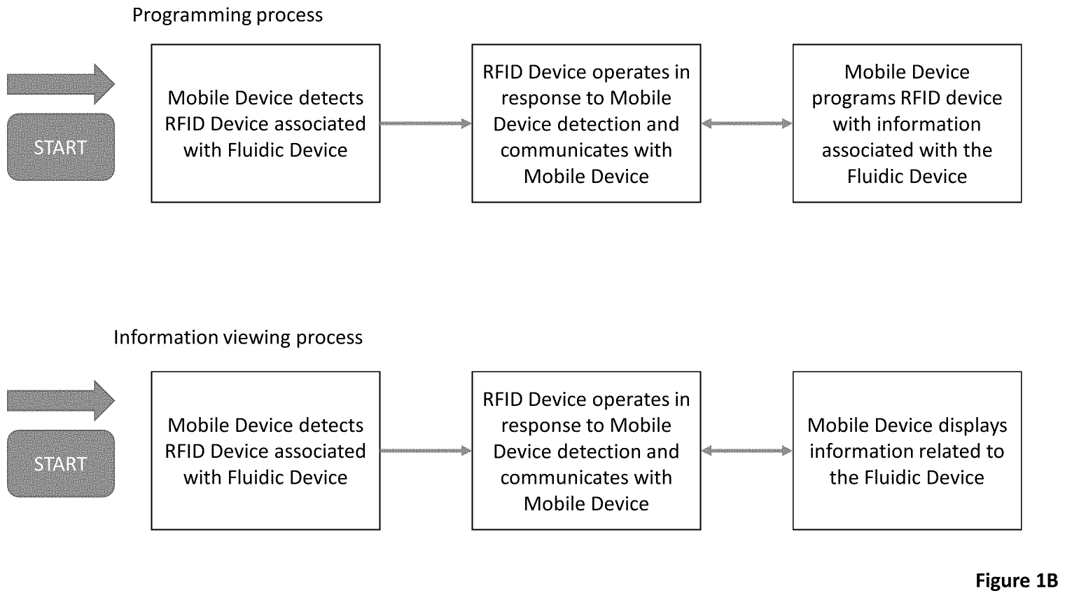

FIG. 1D is an operational block diagram for the test system of FIG. 1A.

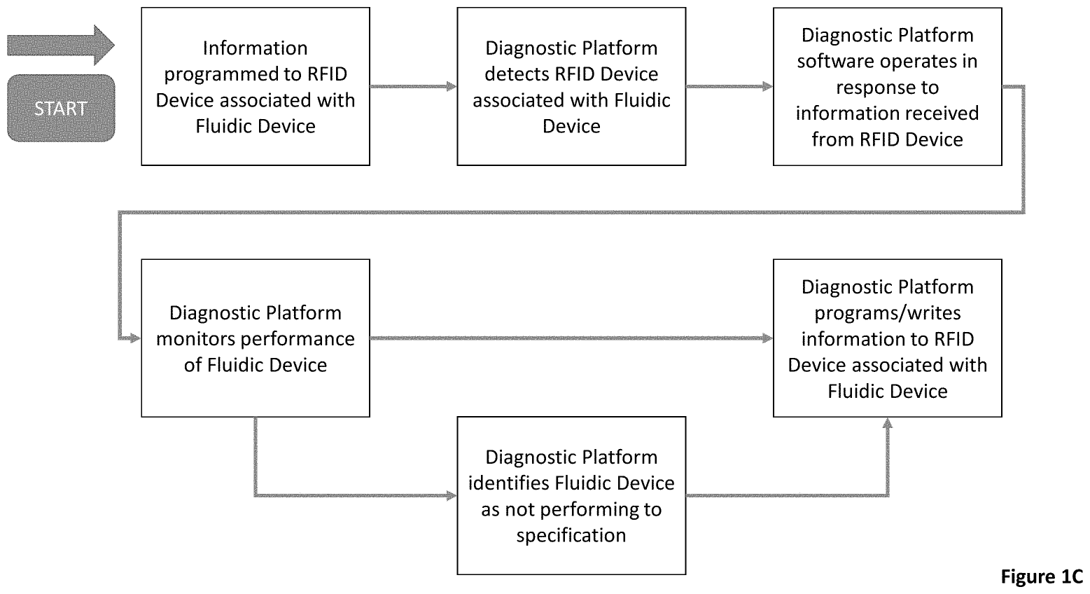

FIG. 1E is a schematic block diagram illustrating the test system having multiple fluidic devices, in accordance with one embodiment of the invention.

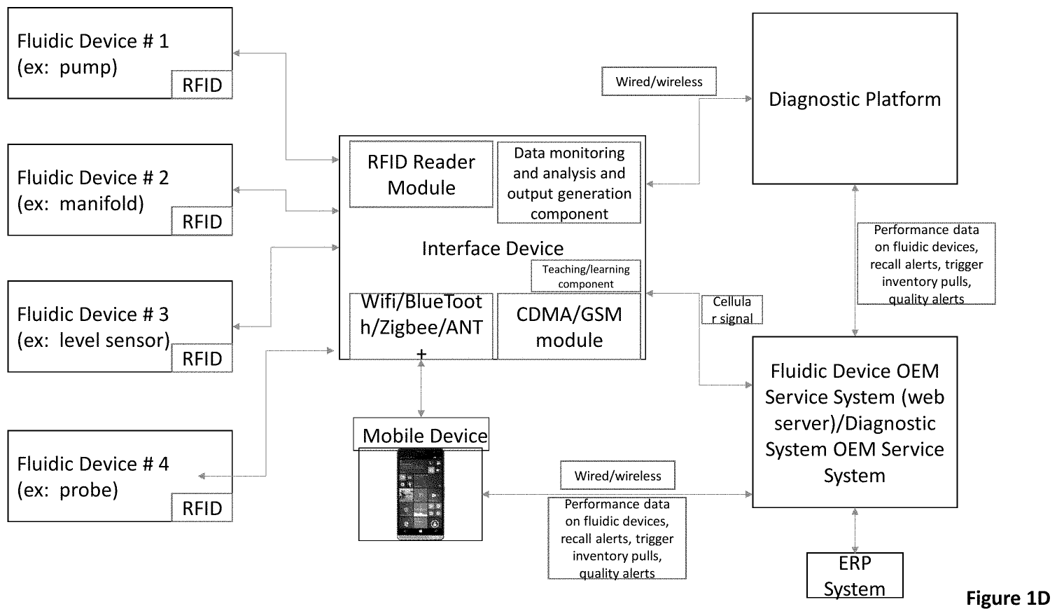

FIG. 1F is a schematic block diagram illustrating the test system having multiple fluidic devices, in accordance with one embodiment of the invention.

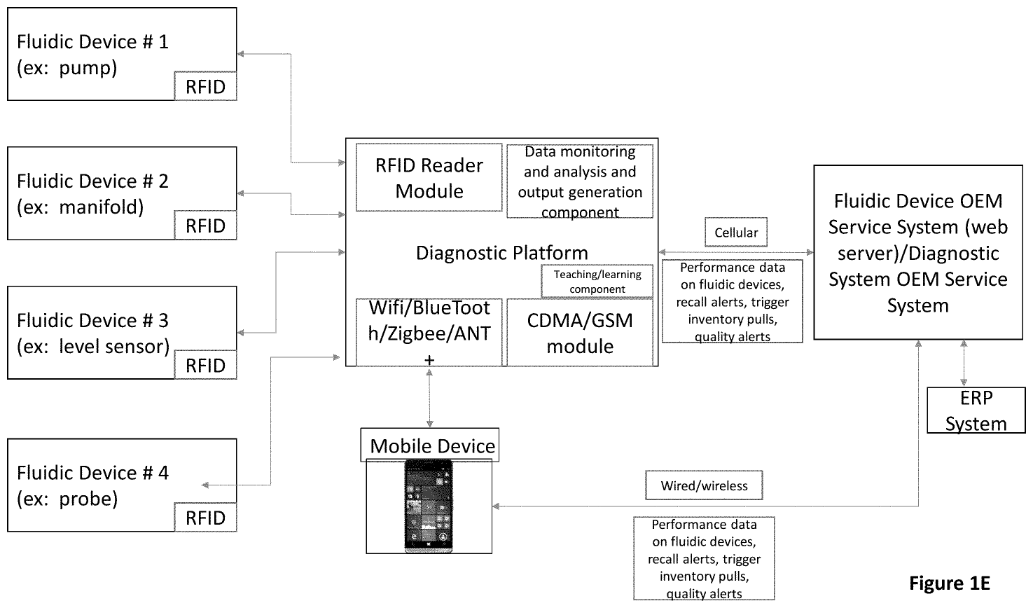

FIG. 2 illustrates a fluidic device having RFID device integration, in accordance with one embodiment of the invention.

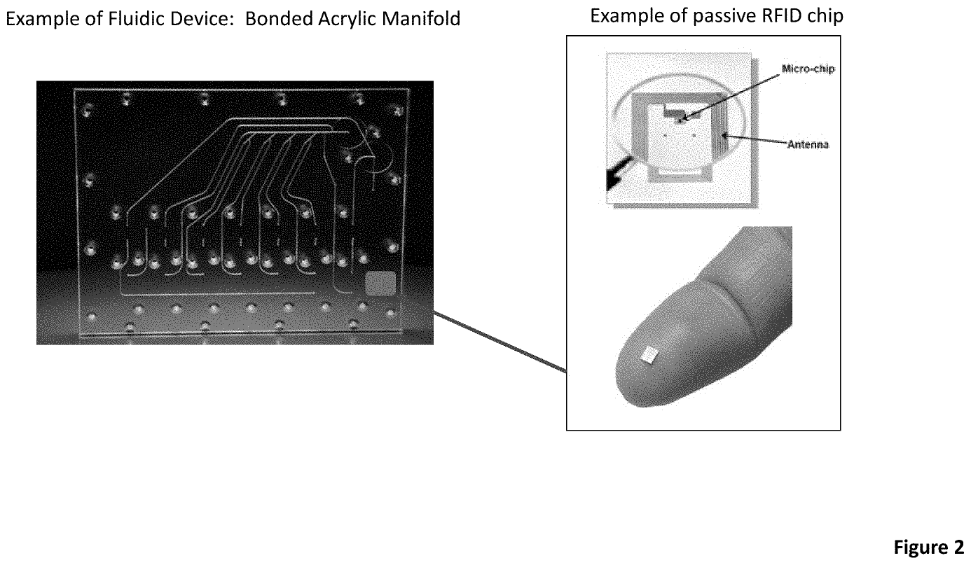

FIG. 3 is an operational block diagram illustrating a method for reading the RFID device of FIG. 2, in accordance with one embodiment of the invention.

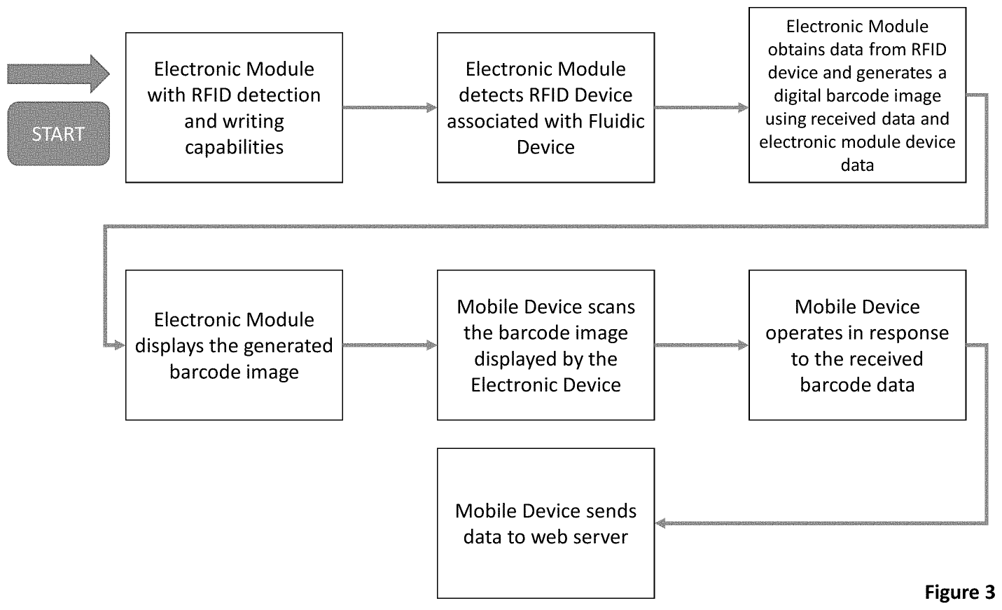

FIG. 4 illustrates a fluidic device having RFID device integration, in accordance with one embodiment of the invention.

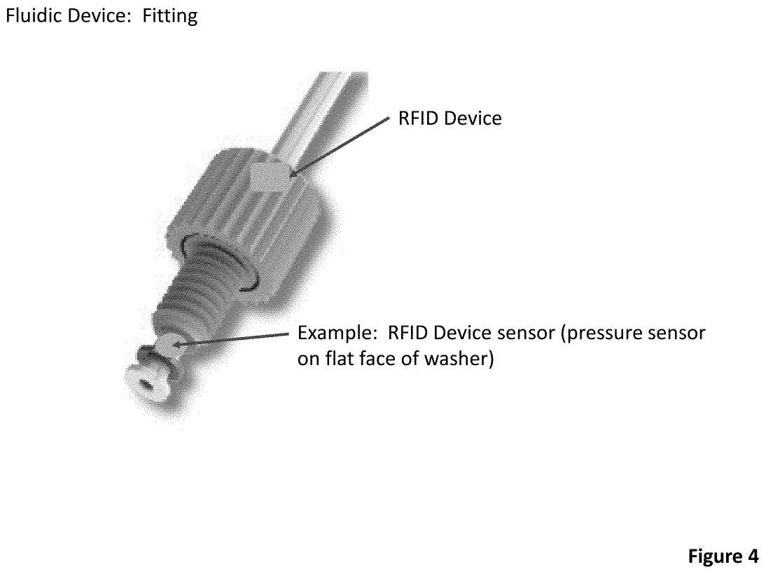

FIG. 5 illustrates a manifold assembly having RFID device integration, in accordance with one embodiment of the invention.

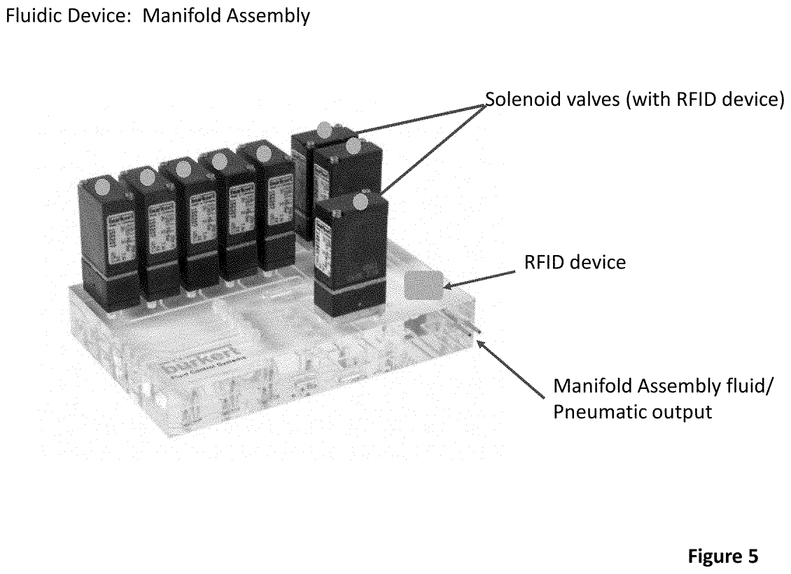

FIG. 6 illustrates a dispense/aspiration having RFID device integration, in accordance with one embodiment of the invention.

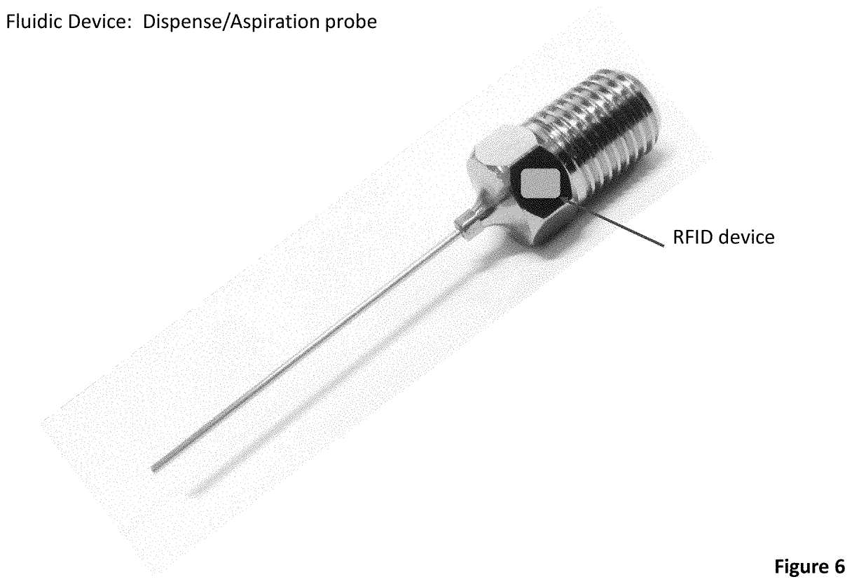

FIG. 7 illustrates a tubing assembly having RFID device integration, in accordance with one embodiment of the invention.

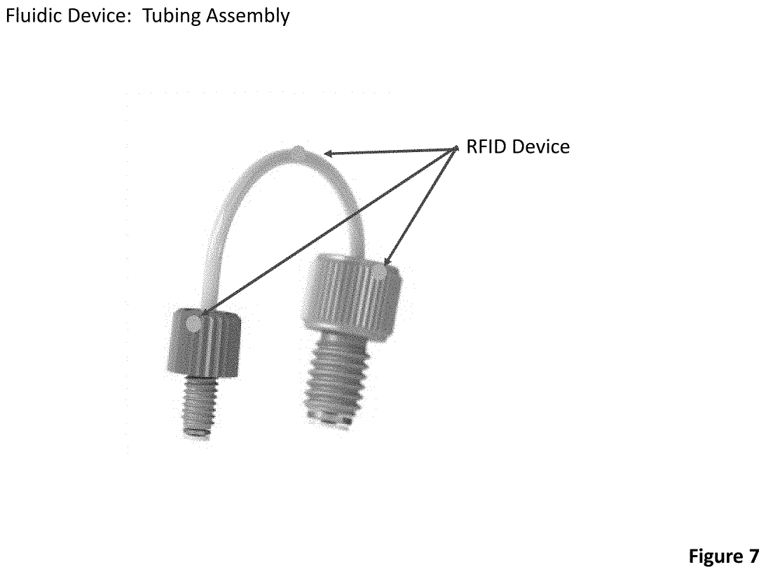

FIG. 8 illustrates a syringe/burette assembly having RFID device integration, in accordance with one embodiment of the invention.

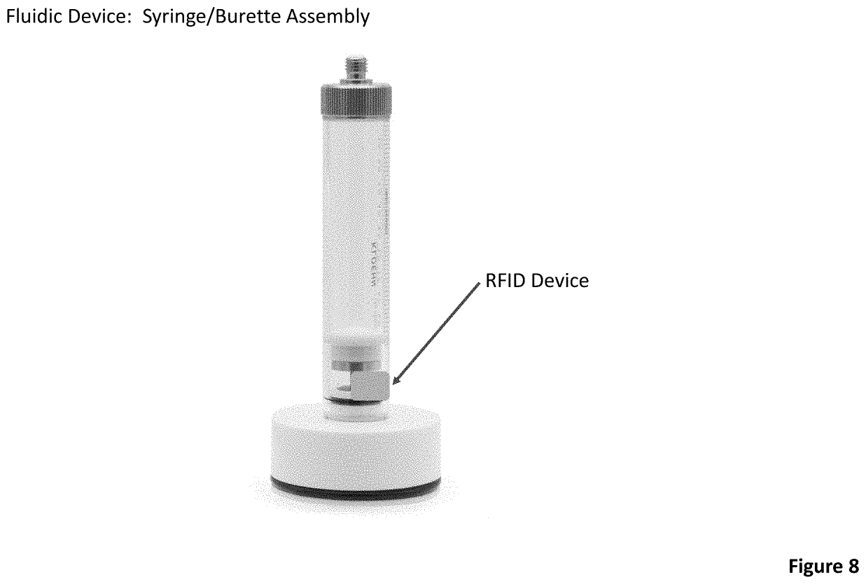

FIG. 9 illustrates a pump having RFID device integration, in accordance with one embodiment of the invention.

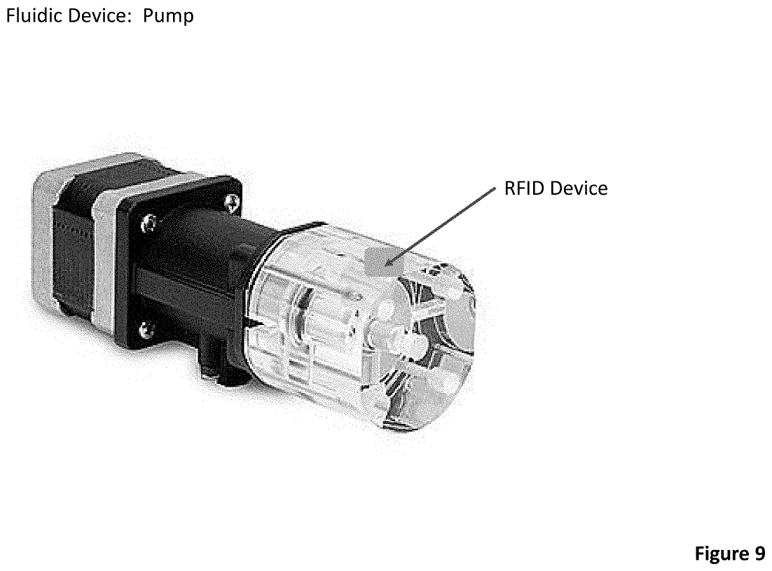

FIG. 10 illustrates a valve having RFID device integration, in accordance with one embodiment of the invention.

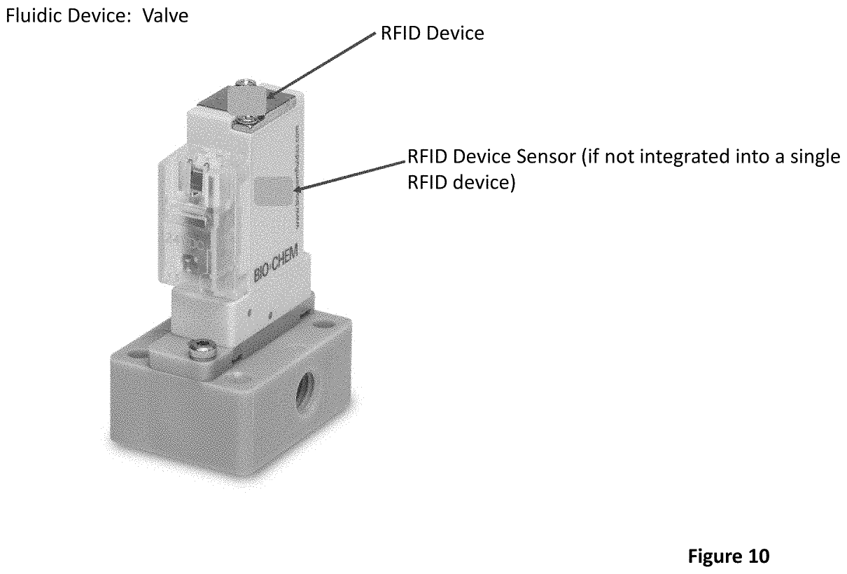

FIG. 11 illustrates a microfluidic assembly having RFID device integration, in accordance with one embodiment of the invention.

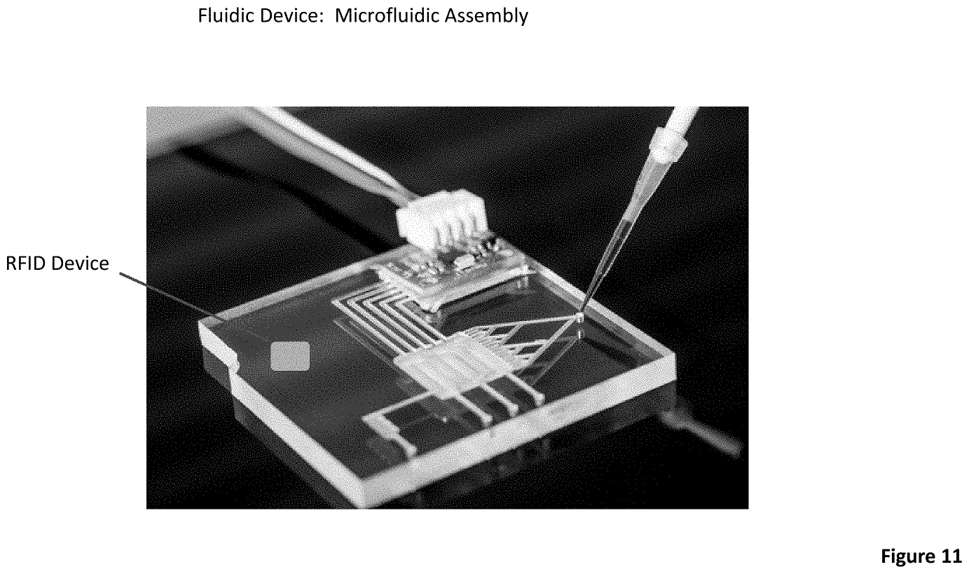

FIG. 12 illustrates a chromatography column having RFID device integration, in accordance with one embodiment of the invention.

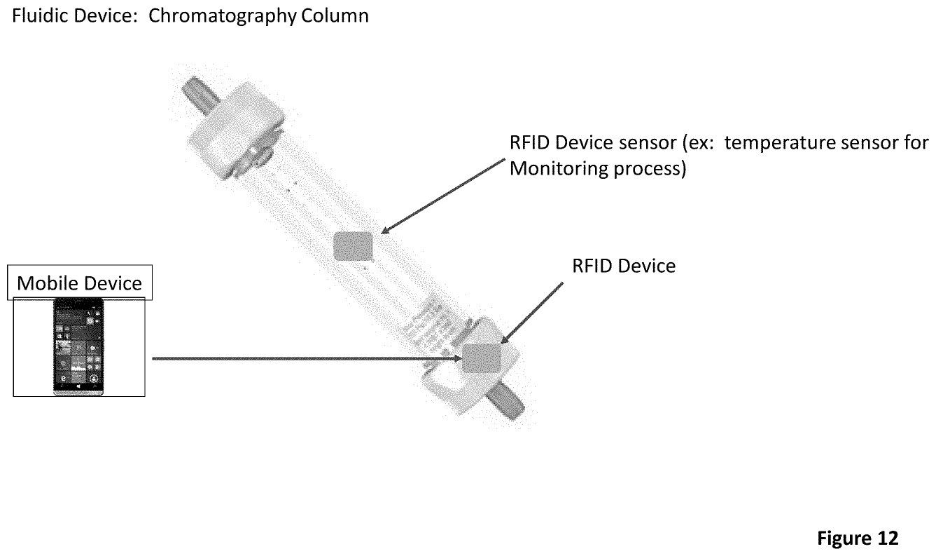

FIG. 12A illustrates a chromatography system having RFID device associated with a column, in accordance with one embodiment of the invention.

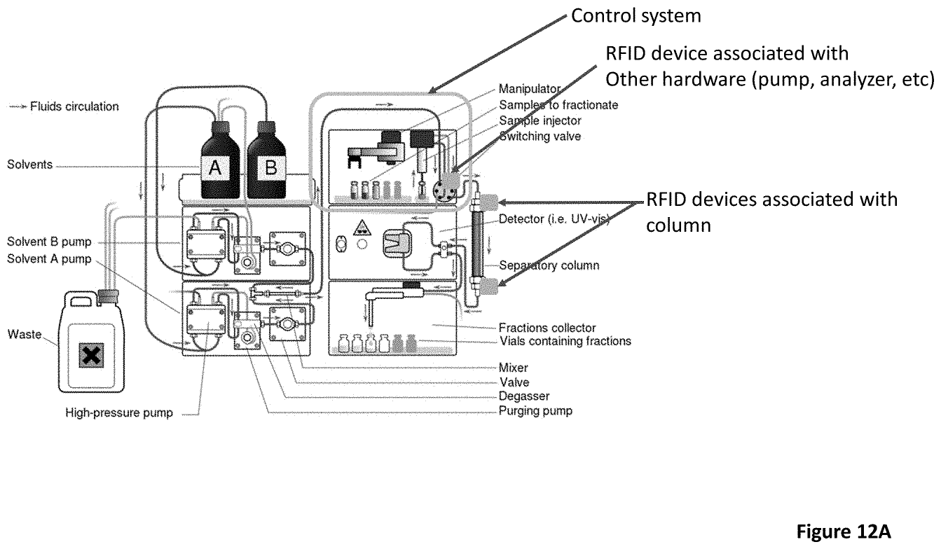

FIG. 13 illustrates a fluid level sensor assembly having RFID device integration, in accordance with one embodiment of the invention.

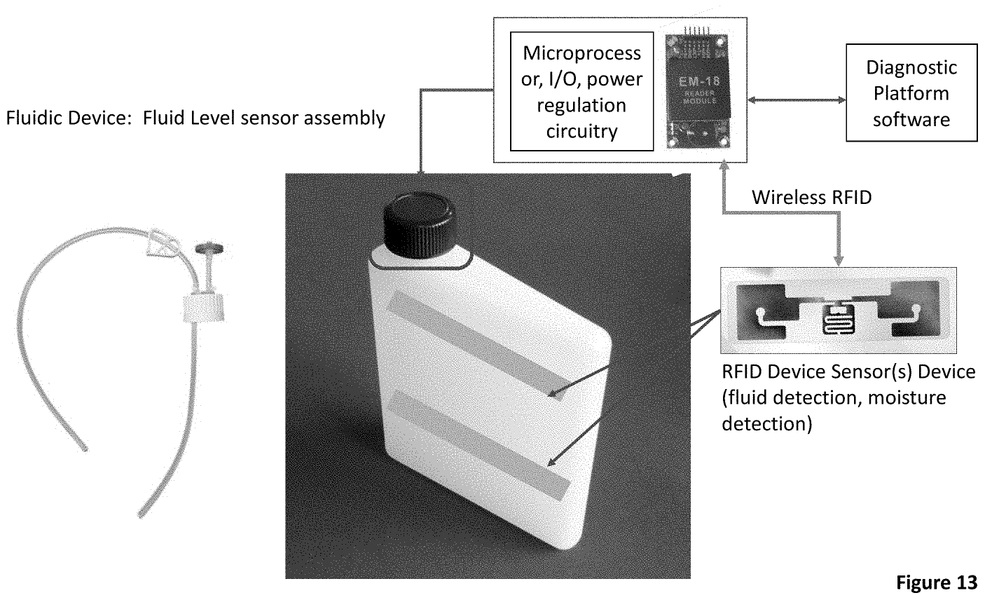

FIG. 13A illustrates a fluid level sensor assembly having RFID device integration, in accordance with one embodiment of the invention.

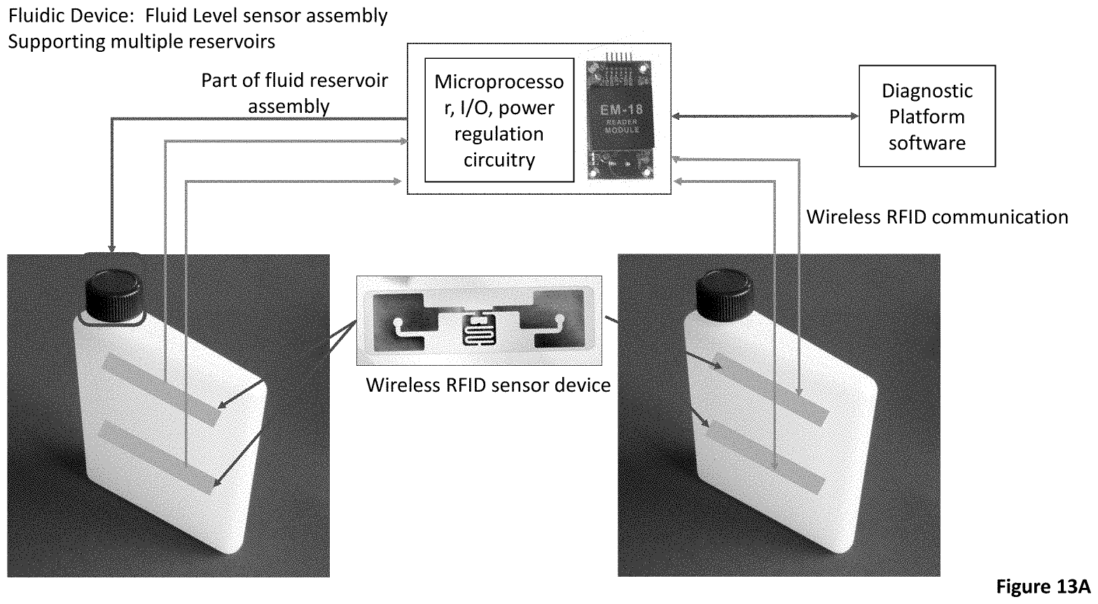

FIG. 13B illustrates a schematic block diagram illustrating a fluid level sensor, in accordance with one embodiment of the invention.

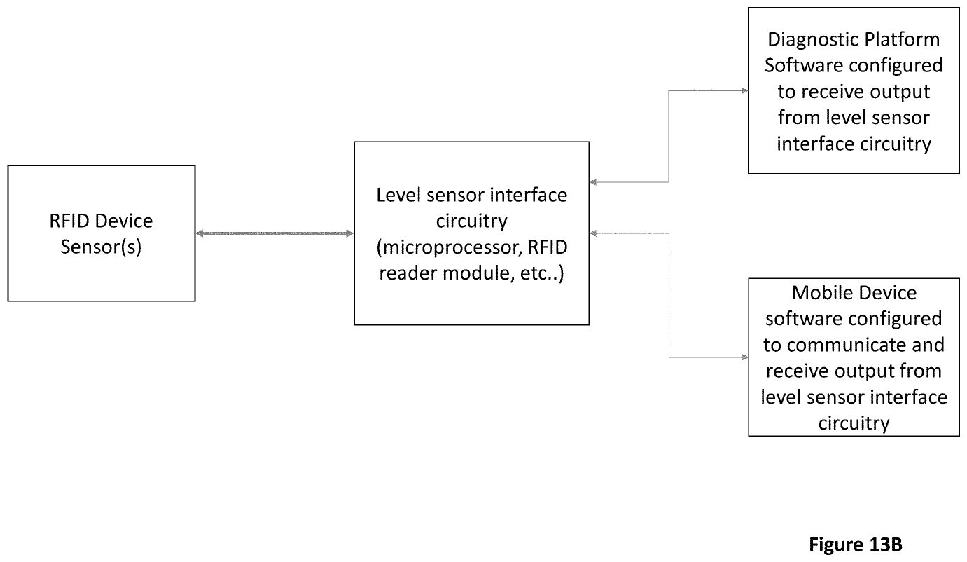

FIG. 13C illustrates a schematic block diagram illustrating a fluid level sensor, in accordance with one embodiment of the invention.

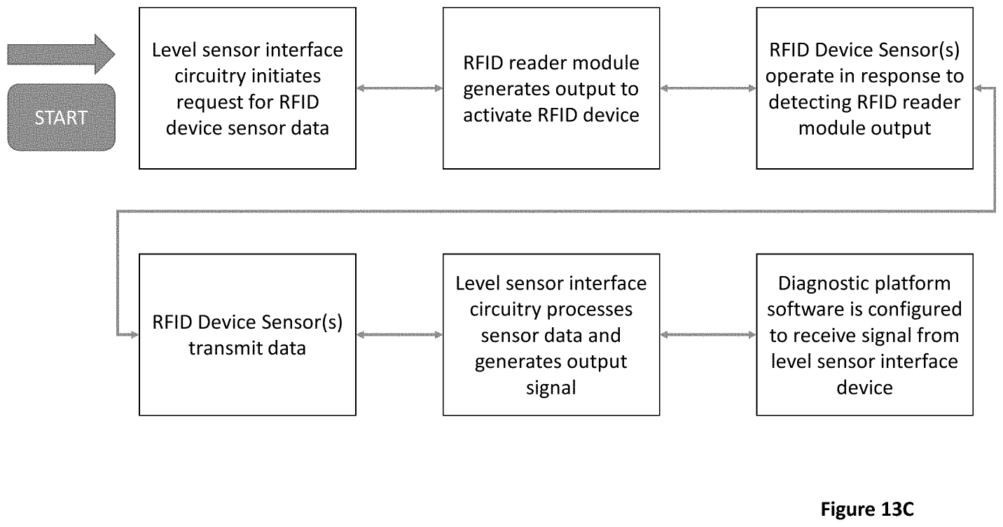

FIG. 13D illustrates a fluid reservoir with RFID device sensor for detecting pressure, in accordance with one embodiment of the invention.

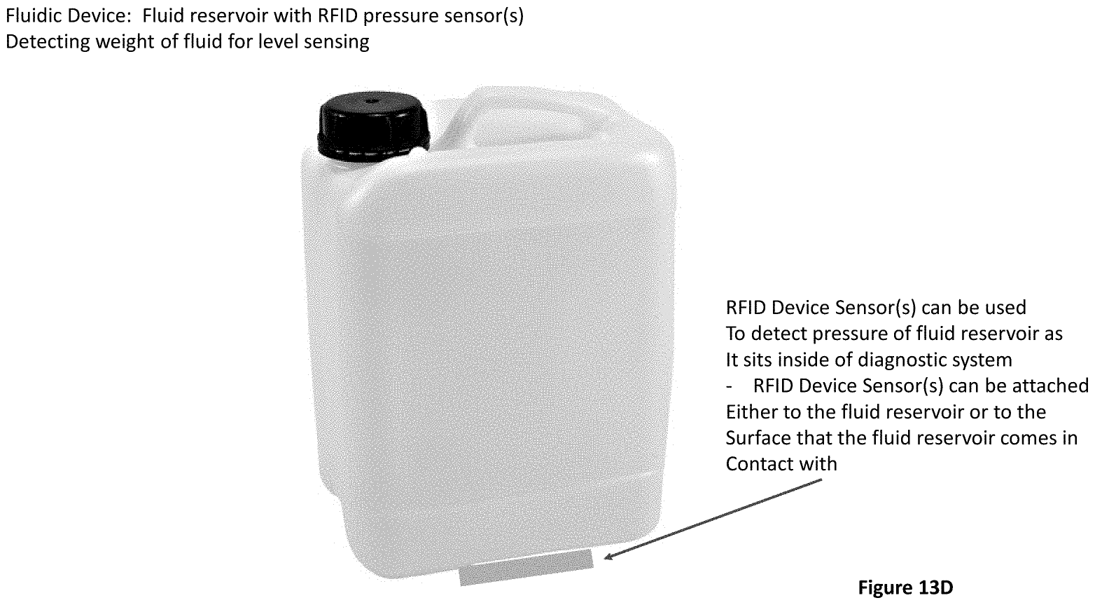

FIG. 14 illustrates a fluidic device (manifold) with a PPG sensor and a RFID device sensor used to activate operation of the PPG sensor, in accordance with one embodiment of the invention.

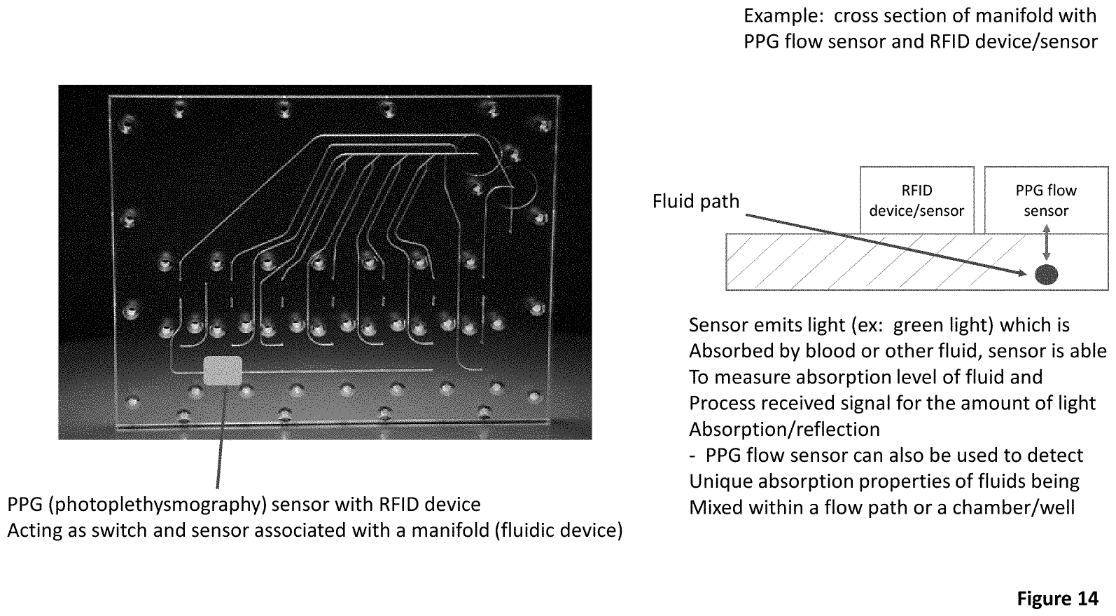

FIG. 14A illustrates a fluidic device (manifold) with a PPG sensor and a RFID device sensor used to detect fluid material properties in a fluid path and/or chamber, in accordance with one embodiment of the invention.

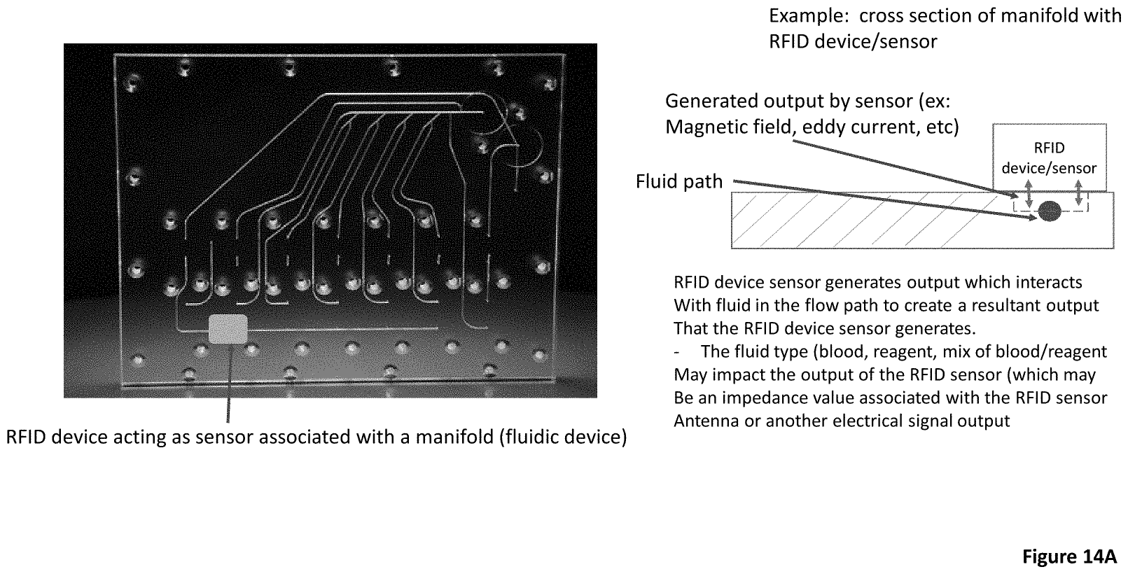

FIG. 15 illustrates level sensor solution for consumable intravenous bag/bio-bag, in accordance with one embodiment of the invention.

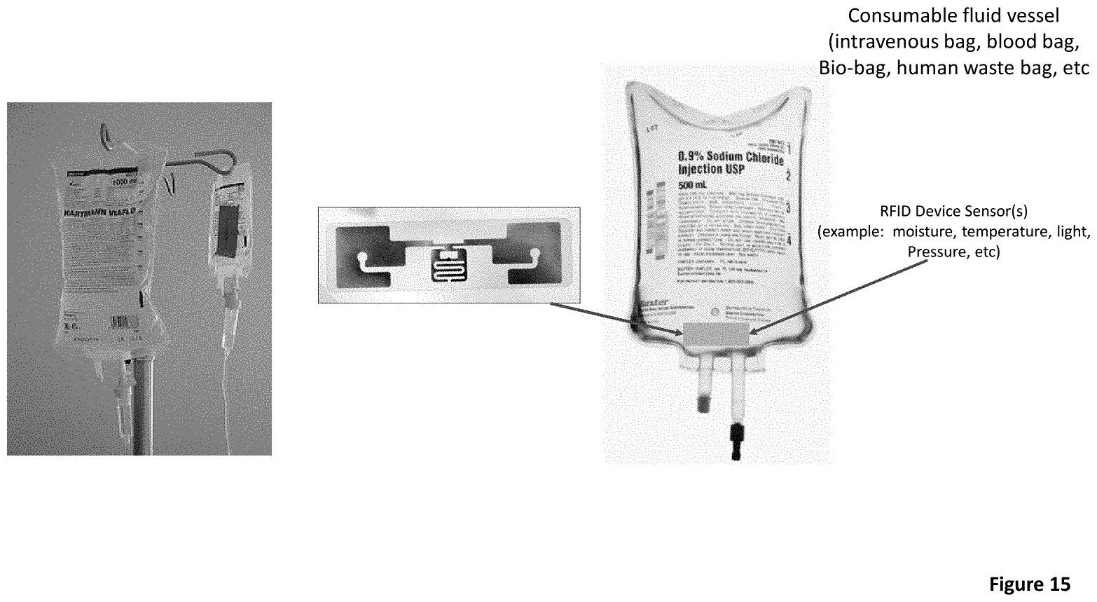

FIG. 15A illustrates level sensor solution for consumable intravenous bag/bio-bag, in accordance with one embodiment of the invention.

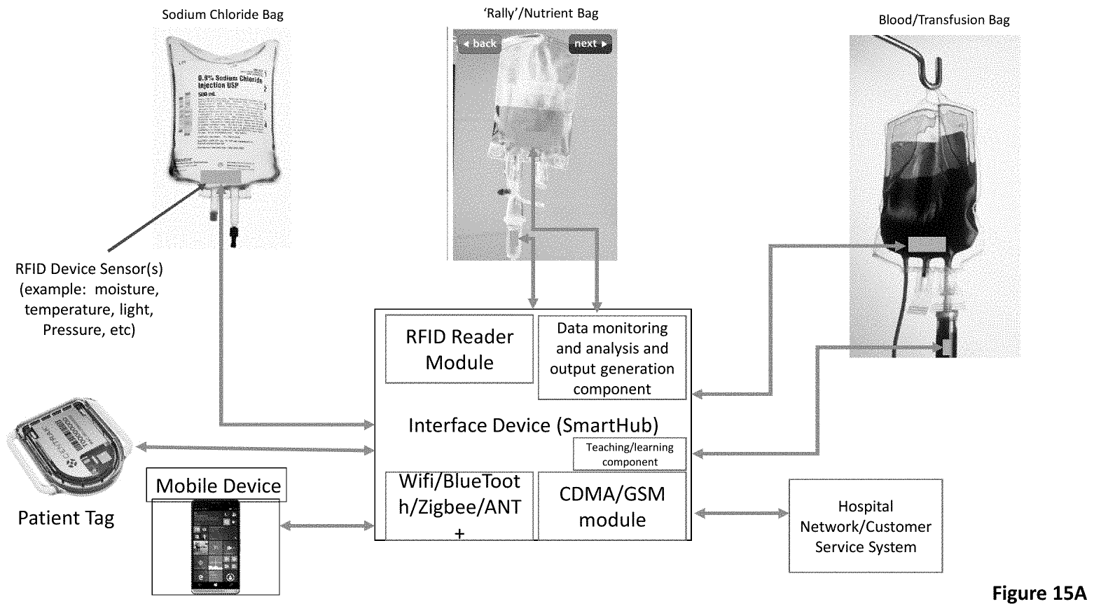

FIG. 15B illustrates fluid bag assembly showing multiple RFID device sensors for redundant monitoring of performance and operation of fluid bag assembly, in accordance with one embodiment of the invention.

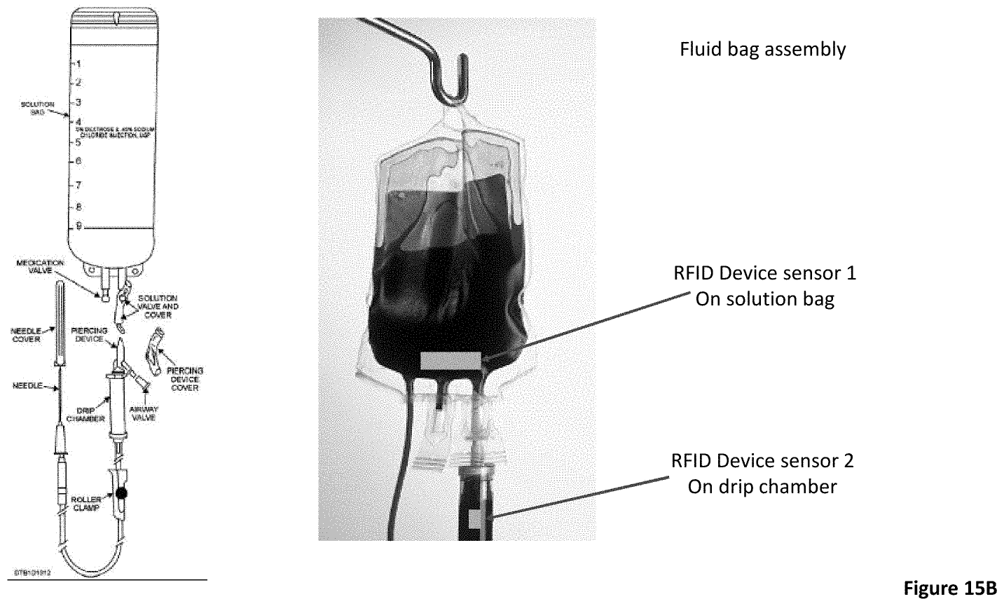

FIG. 16 illustrates a hospital bed with interface device integrated into the bed, in accordance with one embodiment of the invention.

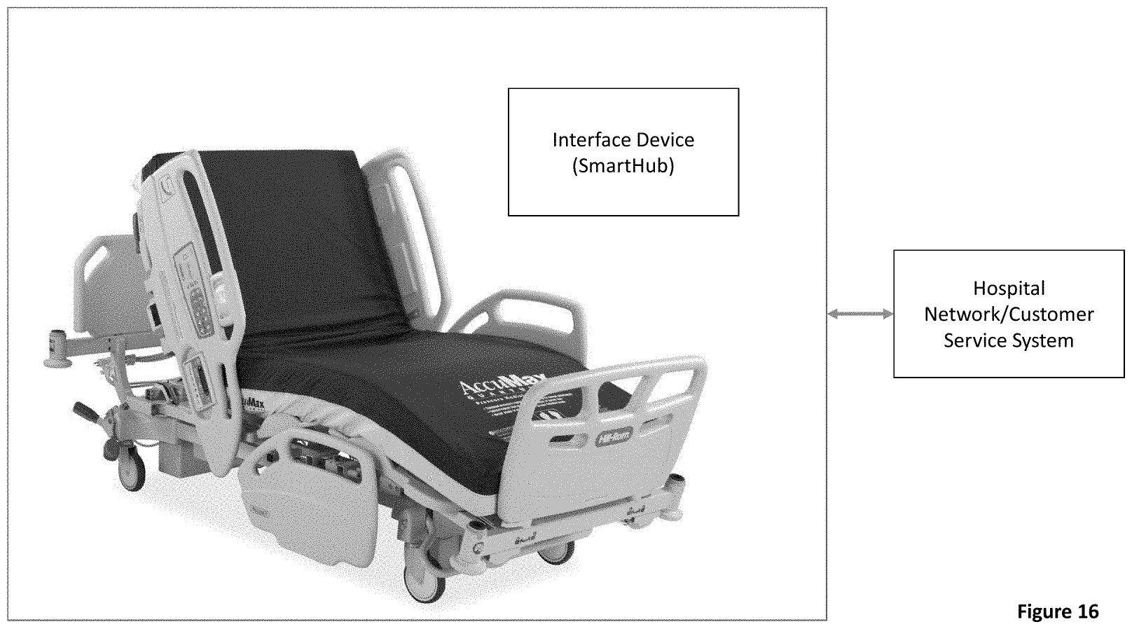

FIG. 17 illustrates a chemotherapy fluid delivery system with interface device capability communication with RFID device sensors associated with fluid bag assemblies, in accordance with one embodiment of the invention.

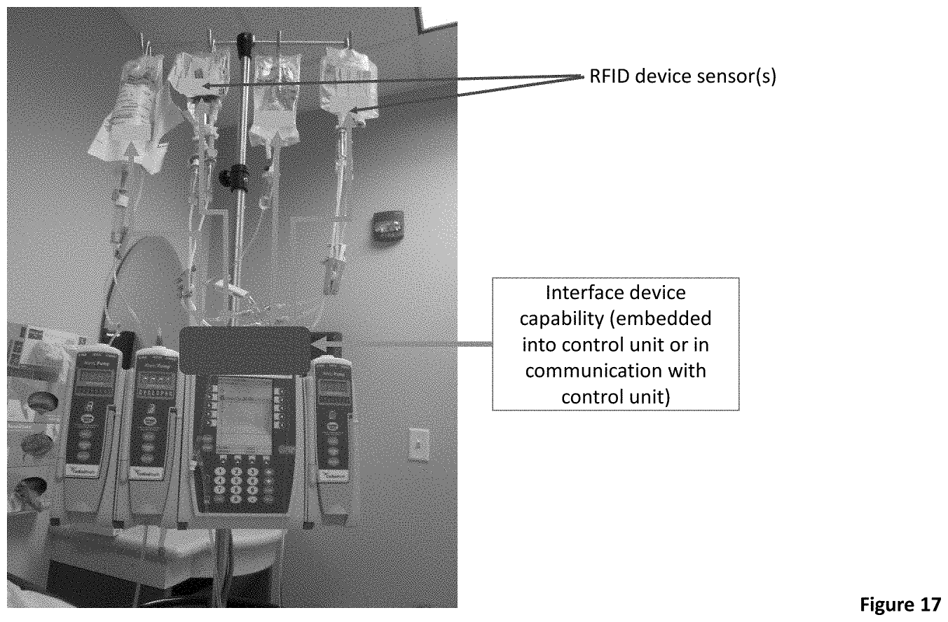

FIG. 18 illustrates fluid vessel cooler with capabilities of interface device for temperature fluid level and quality control monitoring, in accordance with one embodiment of the invention.

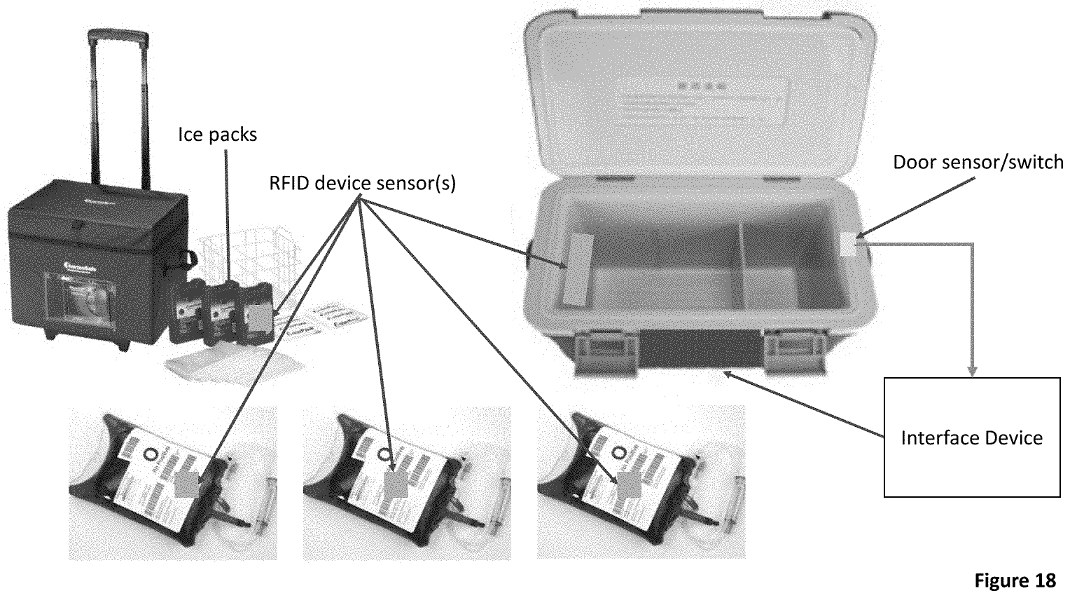

FIG. 18A illustrates system diagram for cooler showing data connectivity to hospital network and external internet network, in accordance with one embodiment of the invention.

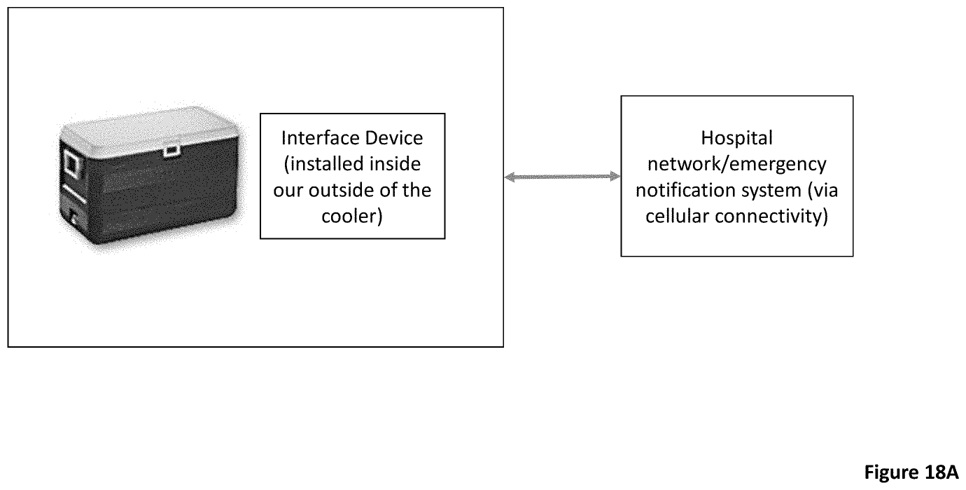

### DETAILED DESCRIPTION

As discussed hereinafter and in accordance with the present invention, a system and method for providing fluidic performance characteristics to a diagnostic platform is disclosed and discussed herein as being applied to fluidic device performance and material traceability and related information.

In accordance with the present invention, it should be appreciated that data may be transferred between devices and/or components via any method and/or device suitable to the desired end purpose. For example, in one embodiment RFID data communication may follow standard methods of communications, such as inductive coupling and/or capacitive coupling. As a simple explanation as to how these two methods operate, inductive coupling involves a reader emitting a magnetic field. When a tag having a chip to be read enters the magnetic field, the chip will vary its antenna response which will result in a perturbation of the magnetic field which can be detected by the reader. It should be appreciated that with regards to HF operational mode inductive coupling devices are inherently short range devices because the strength of the magnetic field decreases sharply with distance from the source. With capacitive coupling, the reader emits a propagating electromagnetic wave. When this electromagnetic energy impinges on a tag, the chip will modify the antenna radar cross section in such a way that the reflected signal containing the information on the chip can be detected by the reader. This is the primary mode of operation at UHF and in the microwave region.

It should also be appreciated that Radio Frequency Identification tags or RFID tags, may be termed “active” or “passive” based on how they are powered. “Active” tags are battery powered and will actively transmit a signal. This type of tag typically has the longest read range (˜100 meters) and are the most expensive due to battery and transmitter cost. “Passive” tags, on the other hand, have no on-tag power source. The energy to activate the chip inside the tags is derived solely from energy emitted from the RFID reader. The read range for this type of tag is limited by the transmitted power density necessary to achieve sufficient voltage for the chip to activate. This type of tag is typically significantly less expensive than an active tag and, in general, will have a significantly lesser range. One other type of tag worth mentioning is a semi-active, or battery assisted passive (BAP) tag. This type of tag includes a batter so the chip will always have sufficient energy to turn on, but it does not have an active transmitter. Since, in general, the limiting factor on the read range of the passive type of tag is getting sufficient power to the chip, the BAP tag has a greater range then the passive tag although at a higher cost and limited life due to the battery.

It should be appreciated that data communications between RFID devices and other devices may be established when an RFID reader module generates and emits electromagnetic energy which is received by an RFID device. The RFID device gathers energy from the reader module and the microchip (IC) embedded inside the RFID device uses the energy to change the load on its RFID antenna and reflects back a signal which is altered in response to the received energy. This altered signal is typically modulated and is known as “backscatter.”

Referring to the Figures, the present invention advantageously provides a unique and novel system and method for overcoming the deficiencies of the prior art regarding the performance and serviceability of a diagnostic platform. It should be appreciated that the components and assemblies (i.e. fluidic devices) that are used in the design and engineering of the diagnostic platform have manufacturing tolerances which dictate their flow performance characteristics. In one embodiment, the present invention advantageously allows for specific flow performance characteristics of a fluidic device to be transferred to a diagnostic platform. This may be accomplished via a test system that is used in the manufacturing (and/or quality inspection) of the fluidic device which physically measures and generates (and/or capture) flow measurement data of at least one flow path (liquid/gas/air flow paths). It should be appreciated that the test system may be configured to measure (and/or capture) a flow characteristic of the fluidic device and generate (and/or capture) flow measurement data responsive to the flow characteristic. The flow measurement data may then be encoded and stored within an RFID device that may be associated with the fluidic device. The test system may also capture multiple flow measurement data points and process this data to generate a processed result. For example, the flow measurement data points may be processed to perform a statistical calculation based on multiple flow measurement data points, generate an average flow measurement result and/or encode the generated average flow measurement test result onto the RFID device. Moreover, the test system may also be configured to encode and store pressure decay test result data, leak test data and diagnostic platform name related data to the RFID device.

In addition to flow measurement data, the test system may also be configured to receive additional information as desired. For example, the test system may be configured to receive data related to the manufacture of the fluidic device. Additionally, the test system may also be configurable to test (and/or calibrate) the performance of the fluidic device and/or test system. For example, the test system may include interface hardware that will allow a desired medium (such as a gas, or liquid) to be introduced into the fluidic device to allow the test system to measure characteristics of the fluidic device, such as pump operation, electrical and/or mechanical performance. Moreover, the test system may also include a software and/or hardware user interface for calibration, control, and/or operation of the test system. Accordingly, the test system may be configured to receive inputs from one or more sensors that are configured to measure the performance of the fluidic device. Furthermore, the test system may be configured to process data from one or more sensors (which may be selectable as desired) to conduct performance and/or statistical analysis. For example, the test system can have inputs from a barcode scanner which can capture information from manufacturing paperwork (i.e. job travelers and/or job routers) where manufacturing lot code information and/or material certification information associated with the fluidic device can be input into the test system. This data may then be encoded and stored within the RFID device.

It is contemplated that in various other embodiments, devices that are used to operate the test system (for example, peripheral devices) may contribute data to the test system, which may then be processed and stored on the RFID device associated with a fluidic device. For example, a test system may rely on a pump for introducing a test media (such as de-ionized water) into a manifold assembly. The pump has an output associated with it which can be measured (such as flow rate, pressure, etc.). The pump may also have interfacing capabilities (and/or built-in sensors) which would allow this output data to be sent to the test system. Moreover, aftermarket sensors may be added to the test system and/or fluidic hardware which may monitor the performance of the pump such as, for example, motor current, inlet and/or outlet pressure, inlet and/or outlet flow. Input data may be any data desired and suitable to the desired end purpose. For example, the input data, which may be related to a device such as a test stand pump, may be received by the test system software and then programmed into the RFID device (or may be directly programmed to the RFID device between the pump device and the RFID device) associated with the manifold, which would allow for the diagnostic platform that will use the manifold assembly the ability to have information related to the inputs that affect the flow performance of the manifold assembly. It is contemplated that the diagnostic platform may use this input data on a RFID device related to the performance of the test system pump to improve the performance of the diagnostic system by comparing the performance of the actual pump, which may be installed inside of the diagnostic platform, to the received test system pump data from the RFID device so it could make sure that the control of the system pump may operate and perform in a similar fashion to that of the test stand pump to help ensure more precise flow performance of the manifold assembly. The diagnostic system would be able to monitor the system pump and control the system pump (or other device, such as a precision pneumatic regulator) in accordance with the target received input data set from the RFID device related to a similar device that was used by the test stand used to test the fluidic device (pump, manifold, probe, etc.).

It is contemplated that in at least one embodiment, the test system may also be able to program a RFID device with additional data that is captured from a device used to functionally test a fluidic device. For example, the test system may receive data from a pump such as a minimum, average, and/or maximum performance capability (such as flow or pressure capability). The test system may use these data points to generate a data set which can be programmed to an RFID device which can be associated with a corresponding performance characteristic of a fluidic device (ex: manifold assembly). One such performance characteristic may be a flow characteristic where the data set may have a minimum input pump pressure with the recorded manifold output flow, an average input pump pressure with the recorded manifold output flow and the maximum input pump pressure with the recorded manifold output flow. This type of data set could be programmed to an RFID device by the test system which may allow the diagnostic platform to have a complete set of data points that could be used by the diagnostic platform and may allow for additional performance control of the system based on using this data since the diagnostic system would be able to expect a flow output of a fluidic device (like a manifold) if the diagnostic system was replicating a similar input system pump pressure into the fluidic device (ex: manifold). If should be appreciated that there can be more than three data point sets used in this instance so the system would benefit from the improved resolution and performance.

It is further contemplated that the test system can also be connected to a manufacturing Enterprise Resource Planning (ERP) system wherein data associated with the fluidic device, such as date of manufacture, material traceability and certification, and component serial number information can also be inputted into the test system where this data can also be encoded into the RFID device. It should be appreciated that the RFID device may be a passive (without battery), semi-passive (with battery) or active RFID device (with battery) and may benefit from either being a low frequency, high frequency, and/or ultra-high frequency device (to include near-field communication/NFC frequency). The RFID device may have a programmable memory as part of the device which can be used to store the output of the test system as described above. The RFID device can take the physical from of a label or microchip device and can also be part of a printed circuit board that has additional data processing and peripheral device interfacing capabilities. Additionally, the RFID device may benefit from having flexible circuitry, allowing for more precise attachment to a fluidic device which may have round surfaces. RFID device may also include devices such as wireless ‘beacons’ that can have the ability to initiate communications by themselves and may also have their own on-board power supply.

It should be appreciated that in at least one embodiment the RFID device may be comprised of at least one antenna, and at least one microchip (such as an integrated circuit). The RFID device may also have one or more memory banks. For example, GEN 2 RFID devices include four memory banks (reserved memory—typically used for storing sensitive data that needs to be protected, electronic product code (EPC) memory—used for storing a unique code associated with the RFID device such as a 94 bit code, tag ID (TID) memory—memory that is used for storing the unique microchip identification code and is typically not a writable memory bank, and user memory—used for applications where there is a need for more memory than what is available by the EPC memory bank). The RFID device may also have the ability to generate a unique sensor code which may be generated as a result in the change in the measured impedance of the RFID device antenna (such as the RFMicron ‘Magnus’ product line) where the sensor code may also be the output of the RFID device based on the sensing capability.

It should be appreciated that the fluidic device may be any type of fluidic device suitable to the desired end purpose. For example, the fluidic device may be at least one of a threaded fitting (metal, ceramic and/or plastic), a tubing assembly which may include at least one fitting, a machined manifold (metal, ceramic and/or plastic), a multi-layer bonded manifold (metal, ceramic and/or plastic), a syringe assembly, a burette assembly, a valve, a fluid reservoir with a bottle cap, an intravenous bag, a blood bag, a fluid bag used for chemotherapy applications, a plasma bag, a bottle cap assembly, a liquid chromatography column, a flow cell, a flow cell assembly, a pump, a dispense probe, an aspiration probe, a fluid heater and a fluidic assembly module which may include a combination of earlier mentioned fluidic devices.

The fluidic device can be configured to associate with at least one RFID device that has been encoded (programmed) by the test system by allowing for the RFID device to be physically attached to a surface of the fluidic device, or by having a physical feature on the fluidic device that would allow the RFID device to be assembled as part of the fluidic device assembly. For example, in one embodiment a thermoplastic manifold may have a machined pocket that would allow for a RFID microchip to be installed and then adhesively secured into place.

Additionally, a fluidic device may benefit from being associated with one or more RFID devices. Furthermore, multiple RFID devices may each use a different specified frequency which would allow for isolation of how data is communicated by the invention. For example, a piston pump may have two RFID devices where one programmed RFID device uses a low frequency (example: 125 KHz) and the other programmed RFID device uses a high frequency/NFC capability (example: 13.56 MHz). In this instance, the low frequency RFID device may only be capable of close proximity data communications and may need to be within ten centimeters distance of a corresponding RFID transceiver device that is associated with a diagnostic platform. The low frequency RFID device that is associated with the pump may include data such as precision dispense performance data of the pump which can be used by the diagnostic platform software to improve the performance or add value to the calibration of the diagnostic platform. The high frequency RFID device (a NFC chip for example) that is associated with the pump may be used only by the OEM of the pump since NFC offers a secure method of data exchange which allows for the opportunity to restrict access to the fluidic device (for example, the pump) data that may reside on the RFID device/NFC chip. The RFID device may also have a dual frequency capability which uses a shared memory such as an RFID label which may have antenna configurations to support UHF and/or NFC communications, but may share a similar memory storage capability.

The diagnostic platform may also be able to take advantage of the RFID device/NFC chip since it allows for bidirectional communication. This would advantageously allow the diagnostic platform to encode information related to the performance of the pump (fluidic device) such as total number of times the pump was actuated and/or specific error codes associated with the fluidic device. This would also allow the OEM of the fluidic device to gain access to information that is not currently available, while also allowing for the information to be precisely identified with a specific fluidic device. The data that is programmed onto the RFID device in this invention may be encrypted (or partially encrypted) by either the test system, the diagnostic platform, or even a mobile device that may have the ability to communicate and program the RFID device.

Furthermore, in one embodiment, the diagnostic platform may have at least one RFID transceiver (or transponder) that is able to communicate with a RFID device associated with a fluidic device that is associated with the performance and operation of the diagnostic platform. The diagnostic platform software is configured to receive and process the data from a RFID device associated with a fluidic device. For example, flow performance data from a RFID chip associated with a dispense probe (made from either metal or plastic, or even a combination of both such as a Teflon lined stainless steel probe assembly) may be utilized by the software of an In Vitro diagnostics (IVD) platform to ‘auto tune’ (calibrate and configure) the performance of the IVD platform in relation to both reagent and sample dispensing operations.

Still yet another embodiment of the invention involves how the diagnostic platform may be able to detect the carry-over performance of a fluidic device, such as a dispense probe. As the diagnostic platform uses the dispense probe, concerns exist about how long the probe can be used before sample (or reagent) residue may risk contaminating other sample fluids (or reagents). This may require a diagnostic system to have the ability to monitor the carry-over performance of a fluidic device, such as a dispense probe by running a self-diagnostic test between sample runs where the self-diagnostic test uses dye and water to measures the amount of dye that has been flown through the dispense probe by looking at concentration levels of remaining dye when dispensing only water. When the diagnostic platform detects a carry-over performance value that exceeds (or is trending towards exceeding) a pre-defined carry-over threshold, the diagnostic platform may write to or program the RFID device associated with the fluidic device a value or indicator associated with the carry-over performance of the fluidic device. Additionally, the diagnostic platform may also write to or program a RFID device associated with the fluidic device a status indicator which can identify the fluidic device as no longer being able to perform within specification where if this status indicator were detected by the diagnostic platform (example: a service technician accidentally installs a probe that has already been identified by the diagnostic platform as not within specifications) the diagnostic platform software would inform the operator of the diagnostic system (via a user interface display message or light indication) that there is a problem with the fluidic device and it needs to be changed with a new fluidic device, in this instance, a new dispense probe.

The invention may also allow critical real use performance data (such as the number of times a probe was used to dispense sample and/or reagent, the number of times the probe was exposed to wash cycles and carry-over testing by the diagnostic platform) to be shared with the manufacturer of the fluidic device (i.e. dispense probe) and the diagnostic platform OEM since the original fluidic performance data which may already exist on the RFID tag (i.e. flow performance data) and even date of manufacturer can now also be compared with the data that the diagnostic platform would write to or program on a RFID device associated with the fluidic device. This would allow the manufacturer of the fluidic device to analyze the data and review opportunities for manufacturing process control improvements based on reliable real world use data which is coming directly from the machine that the fluidic device was installed in.

Still yet another embodiment of the invention allows the diagnostic platform to create an identification code that will be programmed to the fluidic device. For example, a diagnostic platform may benefit (or require) in that a fluidic device, such as a manifold which is installed or that is to be used by the diagnostic platform, may need to be identified by the diagnostic platform in an effort to prevent other diagnostic platforms that may be in the same facility or elsewhere from using the very same fluidic device (for the purpose of preventing contamination between reagents and samples). In this example, the diagnostic platform may look to see if there is an existing identification code on the RFID device associated with the fluidic device. If there is an ID code on the RFID device and it matches or can be accepted by the diagnostic platform, the diagnostic platform may proceed to be used. If there is not any ID code on the RFID device, the diagnostic platform will generate an ID code and write the ID code information to the RFID device. The diagnostic platform may also generate and send a ‘kill command’ to the RFID device associated with a fluidic device which may permanently (temporarily) disable the RFID device. This function may be useful for the invention since the diagnostic system could prevent components that are not at the correct design revision level from being used and could identify these components as non-usable since the RFID device may also contain fluidic device revision information and this revision information can be compared with information that is either stored locally within the diagnostic platform software or stored remotely on a web server and which the diagnostic platform is in communication. This would also help protect the diagnostic system from allowing any non-approved fluidic device components from being utilized in the system.

A mobile device may also be able to have the same capability to send commands to the RFID device (such as a ‘kill command’) which would allow a quality inspector or field service technician the ability to ‘reject’ and/or replace bad parts as needed. The diagnostic platform may also program an RFID device/sensor associated with a reagent bottle (for example, assay fluid) with an identification code which may allow that specific assay fluid to only be used with that specific diagnostic machine. Similar to a fluidic device, the reagent bottle may have a RFID device (or RFID device sensor) with writable/programmable memory attached to it which would allow the diagnostic platform to write data associated with the use of the reagent bottle (such as amount of fluid that is left in the reagent bottle, identification code, last date the reagent bottle was used, status of the reagent bottle as expired or not) to the RFID device. This would allow a partially used reagent bottle to have self-identifying features if the reagent bottle is used again by a diagnostic platform. There are instances where diagnostic platforms may not want to have a partially used reagent bottle used as part of a test so this could prevent this from happening by having the diagnostic system either capture data from the RFID device memory associated with the reagent bottle and/or by getting fluid level data from a RFID device sensor associated with the reagent bottle.

Still yet another embodiment of the invention includes an electronic device that can communicate with an RFID device. The electronic device may include a battery, a RFID transceiver (and/or NFC capability), a microprocessor configured to support both RFID and NFC data communications as well configured to process received data from the RFID device and generate a digital barcode on a display screen associated with the electronic device. The electronic device may also allow for data from a RFID device (which is associated with a fluidic device) to be displayed as a barcode further allowing a mobile device to scan the barcode where the mobile device is configured to either send the data to a web server where the data can be processed and displayed back to the user via a mobile web page, or the data string inside of the barcode can be processed and displayed within a native mobile device software application.

One example of this embodiment would be a solenoid valve having an RFID device, and the electronic device is able to capture data specific to the valve via its communication with the RFID device (for example: pre-determined dead volume characteristics, measured dead volume characteristic, flow characteristics, valve actuation response time characteristics, valve coil resistance value, valve orifice dimension information, environmental operating range characteristics). This same data set for a valve which can be encoded inside of an RFID device associated with a valve may also be utilized by a diagnostic platform software application to improve the performance and accuracy of the diagnostic platform. The electronic device (such as a barcode display module) may allow a service technician who does not have a mobile device capable of detecting the RFID directly to use the electronic device to capture the data from the RFID device, wherein the electronic module would display the received data as a barcode which in turn would allow any mobile device with barcode scanning capabilities to utilize the data. Moreover, the electronic device may benefit from a low power display hardware such as a ‘E-Paper’ or ‘E-Ink’ type display which will not consume any or much power when displaying data. This would allow for the electronic device to be powered off of at least one-coin cell battery.

The electronic device could also be ‘awakened’ or turned on by detecting the RFID device allowing for a minimal user interface requirement for the electronic device. A solenoid valve may also have an RFID device integrated into the electronics that operate the valve, which may also include data processing and data storage capabilities (onboard memory and microprocessor) which have the ability to monitor the use and performance (to include error codes) of the valve and make this information available for transmission via the RFID device to another fluidic device the valve may be associated with and/or the diagnostic platform. Additionally, a valve may be able to use sensing capabilities of an RFID device that would be able to detect the operating temperature of the valve. If the valve were using an active RFID device (power was constantly available), the valve would be able to report temperature or even a status/health message/error message to the diagnostics platform that would allow for easier troubleshooting. If a valve was using a passive RFID device (energized and communicates only when exposed to a magnetic field), a valve could report temperature or even a status/health message/error message to the diagnostics platform that would allow for easier troubleshooting when the RFID device sensor is energized by a RFID reader module. The diagnostic platform could then run a system check and send power to one or more valves which will result in an increase in temperature as the valve coil is energized. If there is a problem with the valve, the temperature may not change, or may not change enough over a certain time period. The RFID device associated with the valve would generate a signal back to the diagnostics system (and/or generate a unique sensor code and/or temperature value) which would be used by the diagnostic platform software to verify functionality of the valve.

A further example of this would be that the RFID device associated with a valve may generate a sensor code output and/or a measured temperature output when the valve is not energized to establish a baseline reading. The diagnostics system RFID reader module may energize the valve for a defined amount of time and then initiate communications with the RFID device associated with the valve which may result in a new sensor code output and/or a measured temperature output. The diagnostic software would be able to determine the ‘health’ of the valve by monitoring the temperature performance of the valve. If the temperature on a second request made by the diagnostic platform to the RFID device sensor returns a similar result as the first request when the valve was not energized, the diagnostic platform software would be able to determine that there is a problem associated with that valve, and then may write/program an error code associated with that specific valve to the RFID device associated with the valve (or even another fluidic device, such as a manifold which has the valve mounted to it). The valve coil may also generate a magnetic field which may also be detected by the RFID device sensor which would result in a change in the RFID device sensor antenna impedance characteristic (or other measured electrical characteristic) allowing for the RFID device sensor to be able to monitor the performance of the valve which the diagnostic system (or other machinery) would be able us as part of system self-diagnostic checks or service technician field maintenance.

The diagnostic system software may also be configured to identify a specific valve (or any other fluidic device) based on information it receives from a RFID device memory bank (for example: a EPC code, a TID code, a RFID code, or a valve serial number unique to the valve). This fluidic device identification information may be stored (either temporarily or permanently) as part of the diagnostic platform software which could use the fluidic device identification to query a specific fluidic device for information (such as valve temperature). A mobile device (such as a smartphone, tablet (iPad), laptop PC, wearable device such as Google Glass type device with a heads up display capability (HUD), a handheld RFID reader device, a wrist type wearable such as a watch device like the Apple watch or Samsung Galaxy Gear device) may also be able to communicate directly with an RFID device associated with a fluidic device, negating the need for the electronic device (electronic module with display capability) as described above.

In this embodiment, the mobile device has the ability to transmit data it receives from the RFID device to a web based platform where the data from the RFID can be processed for further evaluation. For example, this captured data may be used to provide feedback to the OEM of the fluidic device (or the OEM of the diagnostic platform) where the feedback is used to address manufacturing quality and process improvements as well as fluidic device supply chain improvements. The mobile device may benefit from direct communications with a RFID device where a native software application may be used to control the NFC reader/writing capability, or the mobile device may also have a web browser software application (such as Chrome) that allows the mobile device to have control over the NFC reading/writing capability through a web application (such as using HTML5 capabilities with the CHROME web browser). The mobile device may also offer the ability for the manufacturer of the fluidic device (or a person who is servicing the diagnostic platform) the ability to write to or program information into an RFID device associated with a fluidic device, allowing for information such as test result data and inspection data that can be entered into the mobile device and then transferred over to the RFID device (as well as data that may be generated by the mobile device such as GPS location information, manufacturer company name for the fluidic device, and/or mobile device user information which may be used as part of manufacturing inspection verification where the mobile device is able to confirm where a mobile device was used to program the RFID device).

For example, a quality inspector involved in the manufacturing of a fluidic device may use a mobile device (or a computer with communication capabilities with the RFID device) to record inspection measurement results on a user interface of the mobile device. The mobile device may also offer the ability for the quality inspector (or manufacturing personnel) to digitally sign and/or authenticate themselves using a fingerprint sensor or facial recognition software capability associated with the mobile device which would allow for a verification of inspection data which could also be written to or programmed to the RFID device associated with a fluidic device the inspector was evaluating.

A further example of this would be in the instance a fluidic device such as a solenoid valve was being tested manually to verify the actuation of valve internal components. The person who is performing the valve actuation test would be able to certify that the test was performed and this test certification information (which may also include a unique date time stamp) could be transferred to the RFID device associated with the valve directly through the mobile device. The mobile device may provide a user interface specific to a test and/or inspection process associated with the fluidic device. The mobile device may first identify the fluidic device type by interacting with a RFID device associated with the fluidic device, and based on this interaction the appropriate test protocol information and quality inspection information may be presented on the display of the mobile device. The information may be customizable (by a user or by design) to display all of the information or only certain information. This user interface may allow the quality inspector to fill out forms and to check boxes that are related to the testing and inspection of the fluidic device.

Once the required user interface options are filled out, the quality inspector may authenticate themselves as described earlier and then they may be prompted to again interact with the RFID device associated with the fluidic device to transfer the test/quality inspection certification information to the fluidic device. The mobile device may receive initial instructions from the RFID device to open a mobile web page (or a native software application) associated with the fluidic device which may have the test protocol information or quality inspection form. The RFID device may include a web address (URL) to identify which specific webpage the mobile device may access. The RFID device may also contain log in credential information (or at least a portion of log in information) related to accessing and using a software application on the mobile device as well as getting access to a website. The mobile device may also benefit from a cable that would have a RFID reader module associated with it where the cable would allow for closer read capabilities of RFID devices that are close together (for example: valves that are mounted close together on a manifold assembly). This would allow a mobile device to more easily get access to RFID devices that are close together.

The invention also includes the ability to offer a solution for field service technicians that service and maintain diagnostic platforms (to include end users of a diagnostic platform) which do not have an RFID device transceiver capability, the ability to benefit from the invention by way of the using a mobile device to communicate information that exists on a RFID device associated with a fluidic device to a corresponding diagnostic platform either providing a type of peer-to-peer (P2P) communication method where the mobile device performs as the in-between device to facilitate communication. For example, in one embodiment, a mobile device can communicate with a RFID device associated with a fluidic device and then (if needed, process the received data) transfer the data to the diagnostics platform directly via a wired or wireless communication method, or by using a network solution method where the diagnostic platform is connected to either a wireless network and/or the internet, and where the mobile device is also connected on the same wireless network and/or the internet and is able to communicate the received data from the RFID device associated with the fluidic device via the wireless network (which may be a local area network associated with the facility that houses the diagnostic platform) and/or an internet connection to the diagnostic platform.

An example of this is a service technician who needs to replace a syringe assembly in a diagnostics machine existing in a laboratory that does not have the ability to detect the fluidic performance characteristics residing on the associated RFID device for the syringe assembly. In this instance, a service technician may use their mobile device to detect the information on the RFID device associated with the syringe assembly, then their mobile device would either prompt the service technician to establish a communication link directly with the diagnostic platform (if one is not already established automatically), or prompt the service technician to communicate the received data from the RFID device to a local area network computer/server which is also in communication with the diagnostic platform and the mobile device, and/or prompt the service technician to communicate the received data from the RFID device to a remote web based server which is also in communication with the diagnostic platform and the mobile device.

As describe above, the use of a mobile device to communicate data that exists on a RFID device associated with a fluidic device to a legacy diagnostic platform allows for diagnostic platforms that have been in the field for many years and that are FDA regulated, the ability to benefit from the invention without requiring new hardware retrofit additions (and possibly only software updates which may reduce risk for validation related to any FDA approval requirements, if needed).

Still yet another embodiment is the use of RFID devices (which may have built in sensor capabilities) to improve how fluidic devices are assembled or even assembled into machines such as diagnostic platforms. For example, a passive RFID device can become part of a tubing assembly where the RFID device has a pressure sensing capability. When the tubing assembly is installed into a diagnostic platform, the fitting that is part of a tubing assembly may excerpt a force/pressure onto the a RFID device with a pressure sensing capability where the RFID device is positioned in between the sealing surface (some examples include face of a flared/flanged tubing assembly, face of a ferrule component, face of a flat bottom fitting) and/or the component that is configured to receive the tubing assembly (or other fluidic device), such as a manifold assembly with a threaded port that matches with the threads on the tubing assembly fitting.

The RFID device may be a passive RFID device, and in this example, if the passive RFID device was in the presence of (and detected) a magnetic field during the installation process of the tubing assembly into the manifold, the RFID device would be energized (powered to work provided by the magnetic field which could be generated by a RFID transponder associated with a diagnostic platform, a mobile device, or another device separate from the diagnostic platform) and would also be able to detect the pressure this is exerted from the assembly process. The pressure sensed by the RFID device could be stored locally on the RFDI device (and may be some value such as a resistance value, impedance value, inductance value, voltage or current level change of the RFID device) where the sensed pressure value may also be obtained by the diagnostic platform and/or a mobile device which is configured to receive data from the RFID device. This data could allow a person installing a tubing assembly to verify that the tubing assembly was installed per recommended guidelines to make sure that the product was not overtightened.

A further example of this would be a service technician installing a tubing assembly into a manifold that is part of a diagnostic platform. The service technician would have a smartphone (or wearing a device like Google Glasses or other heads up display technologies) that would be configured to detect the output of the RFID device during the installation process (either in real time during the assembly or after the assembly was completed). This would allow a service technician the ability to verify that the assembly was done as required, for example within a recommended torque range associated with the assembly. The RFID device would also be able to transmit data to the service technician's mobile device where the received sensor data could be processed (if needed) and then displayed as a torque value or force value.

In one embodiment, the diagnostic platform may also be configured to receive sensor data from an RFID device. For example, a diagnostic platform may periodically or continuously generate a magnetic field that would energize at least one RFID device associated with a fluidic device (or another part of the diagnostic platform). This would allow the RFID device with a built in sensing capability (such as pressure sensing) to output a signal associated with a sensor value (such as pressure data that is being sensed or pressure related data that is stored in memory on the RFID device) to the diagnostic platform. The diagnostic platform software would receive this information and be able to confirm if there are any values that are outside of any pre-determined/stored threshold values residing in the system software. For example, if a RFID device reported a pressure value (or impedance value, or inductance value associated with an applied pressure) that was outside of a pre-determined system pressure related value, the diagnostic platform software could notify a service technician automatically, stop the use of the machine, log the reported value in system memory, identify the fluidic device associated with the reported value via a user interface screen of the system, and/or write information about the reported value back to the RFID device associated with the fluidic device (such as date/time, error code, pressure sensed value that may have been the result of data processing performed by the diagnostic platform).

In accordance with another embodiment, a fluid reservoir may also benefit from the use of RFID devices as the invention describes. For example, a diagnostic platform may have a reagent bottle which supplies reagent. Instead of using a mechanical level sensor solution (such as a float switch which is prone to failures), the invention may benefit from having one or more RFID devices with sensing capabilities attached to the outside of the fluid reservoir (for example, a plastic bottle with a bottle cap assembly) where the RFID device has the ability to detect the presence of fluid and/or moisture with respect to the detection zone capabilities of the sensor (for example, the zone may be a portion of the length and/or width of the antenna). The bottle cap component could be made up of a sub-assembly where there is a circuit board (interface device), a microprocessor, hardware interface options such as a UART, SPI, USB, IIC, etc., that would allow for software updates to the microprocessor as well as signal and data transmission outputs to the diagnostic platform, and an RFID reader (transceiver) module that would be connected to the microprocessor and would allow for the communication link to be established with the RFID device sensor(s) that are associated with the fluid reservoir.

In this example, the microprocessor would initiate the query for sensor data through the RFID reader module to the RFID device(s) that are set up to detect the presence of fluid and/or moisture that exists inside of the fluid reservoir. The RFID reader module may generate the required magnetic field (using at least one antenna) to energize the RFID device(s) that sense the fluid/moisture presence, which would cause the RFID device to output a sensor code value that is associated with the detected presence of fluid and/or moisture of the fluid reservoir. The RFID module may receive this information and then send it to a microprocessor where the data may be processed to generate the appropriate signal value or data set that the diagnostic machine is configured to receive. This would allow for the invention to be used as a ‘drop in’ replacement for existing level sensing products such as float switches. The circuitry in the bottle cap assembly can either be powered by its own power supply such as a coin cell battery, or may also be powered by the diagnostic system hardware. The circuitry and the RFID reader module antenna(s) for the bottle cap assembly may also be at least partially external to the bottle cap assembly. The circuitry may have the ability to communicate either wired or wirelessly with one or more of a mobile device and/or the diagnostic platform. This would allow for either the diagnostic platform software of the mobile device software platform the ability to update software and capabilities of the circuitry firmware and the RFID reader module firmware to support future functionality.

The antenna(s) for the RFID reader module may be placed in close proximity to the RFID device sensor(s) to help ensure the communication link is robust, if needed. The microprocessor and the RFID reader module may exist as part of the same electronic module. The microprocessor firmware may be configured with RFID device sensor unique identification codes which it can use to identify a specific RFID device sensor with a specific fluid level based on the physical location of the sensor on the fluid reservoir. For example, in one embodiment, the RFID device sensor(s) may generate and send data to the microprocessor such as sensor code data (relating to a change in the impedance and/or inductance or capacitance of the antenna), fluid presence data, temperature data, moisture presence/percentage data, RFID sensor identification data, and RFID sensor position data (position is related to where the sensor is physically located on the fluidic device to detect fluid/moisture/temperature). The microprocessor may utilize this information to understand where the fluid level is on the fluid reservoir.

A further example of this would be to a fluid reservoir having a RFID sensor located at the middle of the reservoir. When the RFID device sensor is activated by the bottle cap circuitry, the response from the RFID sensor to the microprocessor could be: Device ID: 123ABC, Sensor code value: 25, Sensor position: middle. This would tell the microprocessor that the fluid level has not dropped below the middle level of the reservoir yet and that the sensor code value of ‘25’ was inside the range of a fluid presence or moisture threshold level in the microprocessor firmware. If the sensor code value was instead sent as ‘10’, and the microprocessor firmware range for fluid detection was between 15 and 25, then the microprocessor would be able to compare and analyze the RFID sensor input and understand the input of ‘10’ to mean that the fluid level has dropped below the middle line of the fluid reservoir. The interface device software/RFID reader module software may also be able to distinguish RFID device sensors by looking at the signal strength/field strength associated with the RFID device sensor and define a range of signal strength in order to filter RFID device sensors not associated with the level sensing assembly. The interface device may also be able to recognize the RFID device sensor by its unique ID such as its EPC code, where the interface device software has been programmed to identify a EPC code with a specific sensor and where the interface device software has a location already stored in its memory associated with a RFID device/sensor.

It should be appreciated that more than one RFID device sensor can be used to add more resolution to the fluid/moisture detection capabilities of the invention. Moreover, the processor(s) associated with the level sensor circuitry (or the RFID reader module) can be configured to accept data from all RFID sensor(s) or the RFID device sensor(s) that are associated with only the specific fluid reservoir the RFID sensors are associated with. Additionally, the level sensor circuitry (or the RFID reader module) can also be configured to accept data (such as, but not limited a response signal) from RFID device sensors that are associated with other fluid reservoirs (and other fluidic devices having RFID device sensors) that are within range of the magnetic field generated by the RFID reader module. This would allow for a single level sensor interface device to have the ability to capture and report fluid level related data from multiple fluid level reservoirs and then report the information to the diagnostic platform. The diagnostic platform software may also be able to directly receive sensor data without having any circuitry inside of a fluid level sensor bottle cap assembly and would benefit from the methods described. The fluid reservoir may also have redundant RFID device sensors for measuring the same area/region/physical characteristic of a fluid reservoir as a first RFID device sensor, which may be used to verify such as things as fluid level, temperature, conductivity, pressure, etc., where at least one second RFID device sensor would be used to generate a second data set to give the system confidence that the metric that is being sensed by the first RFID device sensor (temperature, pressure, fluid level, moisture, etc.) is accurate.

Still yet another embodiment of the invention relates to improving the process controls for liquid and/or gas chromatography applications. The person using a liquid chromatograph machine will go through consumables such as ‘columns’ which are used for such things as protein separation. Temperature control is important in certain applications where improved separation throughput is needed. The invention would allow a person to identify a column to have a temperature range and flow rate (for the internal column plunger speed) associated with it. For example, a person using the chromatography machine would be able to program a temperature range and/or a flow rate to a column which has at least one RFID device (or RFID device sensor) associated with it. The person may also use a mobile device to program the temperature range and/or flow rate characteristics for the column which is configured to communicate with the RFID device associated with the column, so when the column is detected by the chromatography machine RFID reader module, the machine software is able to input the received temperature range/flow rate information specific to the column.

The RFID device sensor (which may also be incorporated as part of a RFID device) would be able to generate a value associated with the temperature and measured flow rate of the column when the machine RFID reader module activates the RFID device sensor. Multiple RFID device sensors may be associated with the column assembly allowing for temperature recordation across the length of the column for improved statistical process control. The received data from the RFID device sensor(s) may be used to modify the performance of a heating control hardware/software which is responsible for heating the column. The temperature data that is captured from the RFID device sensor may also be used by the system to confirm that quality of the separation process in order to improve process traceability and process quality. As mentioned earlier, the machine (chromatography system) may write a status indicator or code (or even a ‘kill command’) to the RFID device which would be used to by the machine to identify the number of times the column (fluidic device) has been used. This information would also be available to a person using a mobile device which could communicate with the RFID device. The RFID device associated with a column may also contain information about the contents that have been ‘packed’ inside of the column (for example, identified as a material code or material name and percentages of material) which can also be information the chromatography system can use to select a specific software program that will run based on the ‘packed material’ that resides inside of the column.

The RFID devices (sensors) may also be used by the machine for real time process flow monitoring where the chromatography machine can periodically energize RFID device sensor(s) associated with a packed column during the separation process to get data throughout the process. A column can also be identified with information that is programmed from a mobile device, where a person who packed the column would be able to provide an electronic signature or secure code that could only be generated by a mobile device (or computer) that the person was using to perform a final inspection certifying the column was packed correctly. This would provide the chromatography machine with information that could be used for traceability purposes and to also ensure that the packed column was verified for quality acceptance prior to using the column in a machine.

A system outside of a chromatography machine which is used to ‘pack’ a column may also be able to communicate with a RFID device associated with a column. For example, hardware may be used to hold the column to accept the media that will be packed. This system may be able to detect the type of media that is being packed into a column (by way of scanning a barcode associated with the media or by manually entry into the system). The system can have circuitry and software that could then write this media summary information to the RFID device associated with the column so the column has traceability for the amount and type of media that has been packed. This media summary information may be entered into this system using a manual entry, barcode entry, or by mobile device with a camera that is scanning information from a label or process paperwork that contain media information. This information could then be used by the chromatography machine as an input for how the machine should perform (for example, pressure the machine should use, fluids that should be used with the separation process, temperature control requirements for the column). It should also be appreciated that a mobile device (or a computer) would also be able to perform the above task of getting data associated with how a column was packed into a RFID device associated with a column.

Still yet another embodiment involves using a RFID device sensor(s) to detect the quality of how a column is packed by associating the output of the sensor with a known good packed column. For example, a RFID device sensor could be used to monitor a characteristic of the column (for example: conductivity, resistance, temperature, etc.), where if the RFID device sensor does not output a value that is within the approved range for the specific column configuration, this can be detected either by a mobile device as part of quality inspection of the column or by the chromatography machine as part of a diagnostic check prior to using the column.

Another embodiment is related to how chromatography columns typically consist of three main components, a tube and two end-pieces (vented cap/bung/plug), one for each end of the tube. When assembled together a reservoir is created in which chromatography media is contained. An RFID chip (RFID Device) could be placed in each of those three parts and the moment the three parts are assembled together with media contained therein, a specific chromatography ‘system’ is created and logged by the RFID chips and associated control system. This ‘system’ could also interact with other components in the chromatography set-up that includes pumps, analysers, etc., that are configured to detect the RFID devices associated with a column. There are many different sizes of chromatography column and many more types of media and separation protocols. A record of column configuration, usage protocol parameters and usage history could all be monitored and recorded, perhaps automatically setting up other equipment to any relevant parameters or preventing misuse.

Still yet another embodiment includes the use of RFID device sensors with microfluidic devices. For example, a microfluidic device (a 3D printed assembly, a thermoplastic assembly, a glass assembly, etc.) could have a RFID device sensor as part of the fluid path(s) where the fluid is part of the antenna circuit for the RFID device so when the RFID device sensor is activated when fluid is present, the RFID device sensor operates in response to the RFID reader module radio wave signal. This would allow for a system to detect if fluid as has been moved throughout the microfluidic assembly if a RFID reader module is trying to activate the RFID sensor which may only activate when fluid completes the circuit to allow the sensor to activate. The RFID device sensor may also be attached to an external surface of the microfluidic chip and may be positioned to below a fluid path of mixing chamber where reagents and sample is mixed. A diagnostic system may emit a light source through the fluid path or mixing chamber and the amount of light that interacts with the fluid passes through the fluid in the microfluidic device and may be able to change the impedance value of the antenna (or other electrical characteristic).

When the RFID device sensor is activated (or if the RFID device is an active device) by a RFID reader module, the sensor output may be able to be correlated with the fluid/sample/reagent that the light source passed through allowing for a diagnostic test to be performed using a RFID device sensor. The RFID device sensor may also include at least one printed photodiode that can be used to detect a light source/wavelength associated with the sample detection process for the diagnostic system. This would allow a microfluidic chip to have data storage and data transmission capabilities related to tests that may be performed on a microfluidic chip assembly. This same microfluidic chip with RFID device sensor(s) may also be associated with patient data by having the diagnostic platform write information associate with a patient sample to the RFID device sensor. A microfluidic device may also have a RFID device sensor which has at least one embedded LED light which may illuminate based on activation of a sensor. This would allow a diagnostic system to use the RFID light to detect an activity or performance of the RFID sensor. For example, the RFID device sensor may detect a fluid temperature or the presence of fluid in a mixing chamber and then illuminate the embedded light.

Still yet another embodiment is for the interface device (with at least one RFID reader module associated with it) to be able to facilitate the communication of fluidic device performance data to a customer service/production management system for anyone of the fluidic device OEMs. For example, a hematology machine can have at least one interface device as described earlier which establishes communications with RFID devices (and/or RFID device sensors). The interface device software can receive data from the RFID devices and may have information stored within internal memory to the interface device circuitry which relates to acceptable performance characteristics of a fluidic device. The interface device software can compare the received RFID device data with the stored acceptable data from fluidic device RFID sensors (or any embedded sensor or communication link for a fluidic device). The analysis may generate trend data that the interface device software monitors and when a threshold is met or exceeded, the interface device may generate an output code (error code, diagnostic code, warning code, etc.) to the diagnostic platform and/or directly (via a cellular connection) to a web server that is associated with monitoring and processing information from an interface device.

The web server application may allow a service technician or a customer service technician to view information received by the interface device, and it may also send the data to an ERP system that is used by an OEM of a fluidic device. The ERP system may use the receive data to take action based on the type of data that was received. For example, a solenoid valve may fail inside a diagnostic system and if the valve has a RFID device sensor able to detect a failure mode of the valve (for example, temperature is not rising based on a diagnostic test), the interface device would generate an output code based on this and send that information either to the diagnostic system for further processing, and/or send directly to the valve OEM customer service website where a web server application would process the information about the valve that was associated with the RFID device sensor attached to the valve (for example, valve number, S/N, number of times used, location of the machine the valve was installed, etc.).

The valve customer service web server application may then send this information along to the ERP system that the valve OEM uses to manage inventory and production. This ERP system would be configured to generate a replacement valve order (new production order or RMA order) based on the received data (such as how many times the valve has been used) and then facilitate the shipment of the replacement valve to a party that would install the valve or ship the valve directly to the location that the diagnostic machine is located.

It should be appreciated that in one embodiment, the interface device may also have information programmed into it that is associated with a location of the machine, or may have a GPS capability to identify the location of the machine automatically. This information can be part of the output code that is generated by the interface device which will be sent all the way to the fluidic device OEM ERP system. The interface device may be contained in a mobile device such as an Android (and/or other) tablet device, and may have external components such as the necessary RFID reader module(s) interfacing with the tablet that may plug into a serial port (such as a USB port) associated with the tablet. The tablet device could then be installed into a diagnostic platform or be located outside of the diagnostic platform.

The interface device can also contain the ability to be programmed (a ‘teaching/learning’ capability) for functionality using a mobile device (for example, a smartphone) and/or a laptop computer. This would allow for the interface device software to be programmed with functionality such as how to interpret and process data received from RFID device/RFID device sensors as well as have information about what machine and/or physical location the interface device is associated (a facility ID code). For example, during the set-up of a liquid level sensing device, a service technician who is doing the installation of the system would be able to first detect information from a RFID device/sensor (such as the EPC code or a unique identified that has been programmed into the RFID device ‘user memory bank’) using their mobile device or laptop computer.

The mobile device or a laptop computer with a barcode scanner would be able to either scan a barcode that contains the RFID device/device sensor unique ID information or would allow the service technician to manually enter the RFID device/sensor into a data field as part of a software application (the RFID device/sensor may have a UPC barcode, matrix barcode, or a QR code on the RFID device or packaging associated with the RFID device/sensor). This information would be temporarily stored in the mobile device application (web app or native phone app). The service technician would then be able to associate unique RFID device/sensor information from a RFID device to a physical product (fluidic device), such as ‘reservoir number 1’ within the mobile device software application using a user interface provided to the user by the mobile device software application. The service technician would be able to link multiple RFID devices sensors to a single fluidic device and save the configuration as a ‘profile’ that can be stored inside of the interface device software (or diagnostic platform software). The interface device software would be able to manage and store multiple fluidic device ‘profiles’ which will allow the software to determine which RFID device sensors are associated with any number of fluidic devices that are part of the diagnostic platform.

Once the configuration process is completed by the service technician, the mobile device would then be able to communicate this information to the interface device software application (or directly to the diagnostic platform software application), which would allow the interface device software to know what physical product (for example, fluidic device) a specific RFID device/sensor is associated with. Upon receiving signals/data from a RFID device/sensor, the interface device software would then be able to generate an output signal (or data set) to the diagnostic platform (or to a customer service system) where the output signal/data set could be used for operation of the diagnostic platform. The interface device may have its own display and user interface to allow for a person to gain access to its software functionality and information. The display may be an e-ink display, LCD display, LED display, OLED display, and/or may also be a touch screen display allowing for the person to interface with the software menu functions of the device. The interface device may also have the ability for a laptop computer or tablet device to be connected to it to allow for a service technician to have access for software updates and programming to the interface device firmware. The display associated with the interface device may be integrated as part of the interface device assembly, or may be external to the interface device assembly where the display may be in wired or wireless communication with the interface device. The interface device can also be configured by a user interface that exists within a diagnostic platform, which would allow a service technician to have direct access to configuring (teaching) the interface device software using the same software that is used to run the diagnostic platform.

The interface device may benefit from security controls which can allow for a tight control of which specific RFID device/sensors are used with the system. For example, the RFID device/sensor(s) may have an OEM specific code or identifier that resides inside of a RFID device/sensor memory bank. The interface device would first look to authenticate the RFID device before accepting data from a RFID device/sensor by confirming that the RFID device code/token is acceptable. The interface device software may also receive data from a RFID device/sensor and then review the received data for an approved OEM code (where the OEM may be the company that sells the system and associated RFID device tags configured to only work with the system). In either instance, if the OEM code is not valid or does not exist, then the interface software will not generate an output that can be used by the diagnostic platform. Data security methods such as encryption can also be employed where the data that is generated and/or resides on a RFID device/sensor is at least partially encrypted and can be decrypted and further processed by the interface device software (or diagnostic platform software). An RFID key fob (using a close range/proximity based frequency such as 13.56 MHz) would be able to be used as a ‘key’ which could allow for additional functionality and capabilities to be unlocked.

For example, an interface device may be sold with a factory default of allowing up to two fluid reservoirs to be detected by the device for level sensing where two ‘profiles’ could be created and supported by the interface device software. If a customer wanted to expand the capabilities of the interface device, the OEM of the interface device would allow for the unlocking or expansion of the interface device by allowing the end user to use a physical device such as a RFID key fob (or USB drive) which has a secured unlock token specific to the level of the capabilities that will be unlocked within the interface device software. The end user would receive the RFID key fob and would place that key fob in close proximity to the interface device circuitry which was configured to detect the RFID key fob/card. The interface device software would detect and read a specific file or ‘unlock key’ from the RFID key fob (or USB flash drive) that would be specific to that individual interface device allowing for the interface device software to unlock more functionality, such as the ability to create another set of ‘profiles’ to support more level sensing of additional fluid reservoirs. This would allow for a unique revenue stream for the OEM of the interface device system and RFID device/sensor(s) since this would allow for the ability to generate revenue by having a single interface device hardware that could allow for expansion and that could support multiple fluidic devices and would also provide system integrity by limiting the amount of data that may be collected by an interface device.

The RFID key fob/card (or USB drive) may also contain a new executable software file that might replace an existing software application file or program that resides on the interface device. Once the interface device capability is updated and unlocked successfully, the interface device would have the ability to either erase the data or files that exist on the RFID key fob/card (or USB flash drive), or in the case of the RFID key fob/card, a kill command could be sent from the interface device to ensure that the RFID key fob/card could never be used again. The ERP system may also initiate communications with a diagnostic platform and/or an interface device which is configured to communicate with fluidic devices. For example, a pump OEM may find out that there is a defect with a batch of stepper motors that are used in the assembly of a piston pump. The ERP system (or the OEM customer service system) would be able to send a request out to all or some of the interface device units and/or diagnostic platforms to query information from RFID devices associated with the piston pump to find out where the pumps with potentially defective stepper motors are located.

This information could be tracked via a RFID device tag that is associated with a piston pump where the RFID device had information about a manufacturing batch code or serial number which could be collected and returned back to the ERP system and/or the customer service system. If the issue was critical, the ERP system could initiate a request to the diagnostic system and/or the interface device which would allow for the identification of the piston pump RFID device as scheduled for replacement or even program the piston pump RFID device with a code/status that would not allow the system to run with the pump installed. This capability may also be performed and managed by the diagnostic machine vendor where they have a similar customer service and ERP system capability used to manage their fleet of diagnostic platforms in the field. The communication between the interface device and the customer service system web server can be either a direct cellular network where the interface device has a GSM or CDMA cellular module. The diagnostic platform may also have the full capabilities of the interface device as part of the diagnostic platform circuitry main mother board.

The system may also use more than one RFID reader module which may be positioned to ensure the proper reading and detection of all system RFID devices/device sensors. A mobile device may also be used to facilitate the communications with either the interface device and/or the diagnostic platform in order to facilitate communications between the diagnostic platform/interface device and a customer service system and/or an ERP system. The access to the customer service system via a mobile device may require bio-metric authentication of a user which can be accomplished using embedded fingerprint sensors on the mobile device, or facial recognition technologies such as Apple ID or Samsung face detection for unlocking a phone. The output of the bio-metric security sensor from the mobile device can also be sent to the customer service system which would in turn authenticate the user and either grant or deny access. It is contemplated that other security access methods may also be used.

Tools that are used for installation and assembly of fluidic devices may also have an RFID device with sensing capabilities (such as pressure sensing) on a surface where force will be exerted during the assembly process. For example, a wrench may be used to assemble a fitting to a manifold or pump. The wrench could be designed to have the RFID device sense the exerted/applied force during the tightening process of the fitting to the manifold. The wrench may also have circuitry and data processing capabilities to indicate when a torque value is reached, for example, the use of LED lights such as red or green to indicate an over torque or acceptable torque measured/applied value to the component being assembled. The wrench may also have a display screen which can be used to display the measured/applied torque value to the component this is being assembled. The RFID device could also be in either wired or wireless communication with the wrench/tool circuitry.

In similar fashion as described above, the RFID device associated with the wrench (or other assembly tool) in this instance can also be configured to communicate with a mobile device. It should also be appreciated that sensing capabilities of the RFID device may include at least one of weight/pressure sensing capabilities, light sensing capabilities, moisture sensing capabilities, temperature sensing capabilities, electrical sensing capabilities (conductivity, resistivity, inductance, impedance, etc.), proximity sensing capabilities, liquid level sensing capabilities, flow sensing capabilities, vibration sensing capabilities. Moreover, it is contemplated that an RFID sensing device may be located and configured to sense when a fitting or other connection becomes loose, wherein if the fitting becomes loose, this can be stored in the RFID sensing device and communicated to a technician for repair.

Still yet another embodiment of the invention involves the use of PPG (photoplethysmography) sensor technology to monitor and detect fluidic device performance characteristics such as flow performance, temperature performance, light absorption characteristics related to fluid flow performance, fluid make up/composition, and carry-over performance. PPG sensor technology is widely used in ‘wearable’ and ‘hearable’ fitness devices such as Fitbit and Apple Watch that are worn on a person's wrist or inserted into the ear. These fitness and health tracking applications use this PPG sensor technology to detect blood flow so the devices can calculate a pulse rate associated with the person wearing the health tracking device.

This invention advantageously describes the use of a PPG sensor to detect real time (or semi-continuous) performance characteristics of a fluidic device that is installed as part of diagnostic platform. For example, a hematology system such as the Siemens Advia 120 blood analyzer mixes patient samples with various assays to perform a wide variety of tests. The diagnostic platform must maintain a status of readiness and cleanliness to ensure that there is no cross contamination between patient samples and reagents that are used to support a wide variety of testing. The diagnostic system will run a wash process in between sample runs which typically is dictated based on a pre-defined wash protocol which may include a wash fluid having bleach and other cleaning fluids used to prepare the fluid lines for a next test. These wash cycles are critical to system performance, but they take time away from performing patient sample runs, which in turn can reduce the revenue opportunity for a facility that needs to have this machine running as often as possible. If a wash cycle is not adequate, there is a hefty expense associated with wasted test reagents and sample scrap, including labor associated with setting up the test.

By using a PPG sensor which has sensor technology capable of detecting the presence and the flow of blood (or reagent, urine, sheath fluid, wash fluids, etc.), the system may be able to monitor the performance of a fluidic device (such as a manifold) where reagent and other fluid (such as blood) paths are used by the system to mix and possibly heat up reagent and test sample fluid in order to perform a test such as counting red blood cells or white blood cells. The PPG sensor may contain at least one of a LED light source (red, green, blue, yellow, purple, orange, etc.) and an infrared LED light source configured to generate a light source (wavelength) that will interact with fluid flowing through a fluid path (or residing in a fluid chamber) and at least one photodiode sensor (or other optical sensor/detection circuitry devices) configured to detect/measure light reflection/refraction/absorption, a processor configured to process received signals based on the light source interacting with the fluid (and fluidic device substrate material) and generate resultant data that can be sent to at least one of the interface device and/or the diagnostic system software.

For example, in one embodiment a PPG sensor may use a green LED light (for example, a 540 nanometer wavelength) to detect the presence of blood sample fluid flowing in a fluid path inside of a multi-layer Acyrlic bonded manifold assembly by way of measuring the amount of green light that is absorbed by the blood sample. The PPG sensor would also be able to measure the percentage of the fluid (such as blood) sample (or reagent) in the fluid path and send this resultant data (for example, a measured light absorption profile) to the interface device software and/or the diagnostic system software where it can be reviewed for quality control purposes to ensure that the mixture/ratio of blood to reagent (the composition of the fluid, or detecting a unique light absorption profile) is per specification for the type of test that is being performed. The PPG sensor may also be able to measure the real time flow of the fluid path it is monitoring, allowing the PPG sensor to provide data to the diagnostic system (or interface device) such as possible flow restrictions or blockages upstream from the PPG sensor which can be used to better diagnose the system and possibly write information about fluidic device performance to a RFID device associated with a component(s) that is responsible for the performance degradation.

The capabilities of the PPG type sensor that is described herein may also be contained as part of a valve assembly (such as a solenoid valve, pinch valve, rotary valve, etc.) where the PPG sensor capability may detect fluid flowing into or out of a valve (or through the valve) and generates an output such as a flow rate of the fluid in the confined space such as a valve body or a tubing assembly (when using a pinch valve). The electronics that are used to actuate the valve may also be combined with the PPG sensor electronics, allowing for a single interface back to a diagnostic platform. This would advantageously provide for a fluidic device (such as a valve) to have dual capabilities such as controlling the flow of fluid while also monitoring the flow of fluid. The PPG sensor can also be configured so it can be attached to fluidic devices such as tubing assemblies where the PPG sensor is able to be safely attached to at least one tubing assembly to perform the fluid flow performance as described herein (flow rate detection, carry-over performance, fluid composition, etc.). The PPG sensor may also be configured to be able to detect multiple tubing assemblies by having multiple light source and detector components as part of the PPG sensor assembly. This would allow for a group of tubing assemblies in a tight space to benefit from a single PPG sensor assembly.

The PPG sensor can also be part of a piston pump assembly where the PPG sensor may be positioned behind a retracted internal piston seal. This would allow for the detection of a leak from a failed seal or plunger assembly where the PPG sensor could be used to detect fluid in an area where there should be no fluid. The PPG sensor can also be used to monitor real time carry-over performance of a fluidic device (such as a bonded acrylic manifold assembly) between diagnostic platform sample runs by monitoring and reporting the measured value(s) of at least one of a sample such as blood, urine, reagent, buffer fluid, calibration fluid, sheath fluid, carry-over test fluid, and wash fluid. Carry-over performance can be detected by monitoring the amount of light absorption in a fluid path.

The PPG sensor may also employ any light source (green LED, red LED, blue LED, infrared light, etc.) which can be used by the PPG sensor which would allow the sensor to actively detect an amount of material in the fluid path (such as residual blood sample or reagent from a prior test) during a wash cycle which is performed by the diagnostic system between sample runs. The PPG sensor can be configured to have its sensing hardware and detection hardware be able to have sampling capabilities that match up with the flow characteristics of the fluidic device fluid path to allow for the PPG sensor to be able to have enough sensitivity and data acquisition resolution to detect residual material that is flowing through the fluid path. Once the PPG sensor is able to detect an acceptable carry-over performance characteristic of the fluid path during a wash cycle, it may generate an output signal or data set to either the interface device software and/or the diagnostic system software which can be used by the diagnostic system to confirm that a next test may proceed.

If the PPG sensor detects/measures an unacceptable carry-over performance characteristic associated with the fluid path during a cycle (for example, a wash cycle) or after a wash cycle, the PPG sensor may process the data and generate an output signal or data set to either the interface device software and/or the diagnostic system software which can be used by the diagnostic system to perform an extended wash cycle which will be also monitored by the PPG sensor creating a process loop that can run automatically until an acceptable carry-over performance characteristic is determined. The system may also use this data to write/program performance data to a RFID device that is associated with at least one fluidic device associated with a fluid path performance. For example, in one embodiment, the diagnostic platform may write an error code or a value associated with a carry-over performance test to the RFID device associated with a bonded manifold assembly so the diagnostic platform may no longer use this fluidic device and to also allow the provided of the bonded manifold assembly to investigate the reason behind the failed carry-over performance test. As described earlier, either the interface device and/or the diagnostic system may send a notification to a customer service system which can utilize this information.

The PPG sensor may also be able to detect a result of sample and reagent being mixed, where the result may be correlated to an actual test that is performed inside of a fluidic device, such as a microfluidic manifold which has mixing chambers that are design to allow the reagent to react with a fluid (such as blood) sample. At least one PPG sensor, or multiple PPG sensors may be used by a fluidic device to be part of a logic circuit that is used for the evaluation of a sample in a fluidic device where the PPG sensor(s) are collecting data such as light absorption/reflection/refraction properties (as well as thermal properties) of the fluid at different stages of a process that is performed during a ‘lab on a chip’ process. The PPG sensors would be able to actively report the measured data to the interface device software and/or the diagnostic system software for further processing and analysis.

The PPG sensor may be powered by an external power source associated with the interface device and/or the diagnostic system. The PPG sensor circuitry may benefit from a communication protocol to facilitate communications with other devices such as a RFID device, an interface device, and the diagnostic platform. The PPG sensor circuitry may also contain status indicators that may be illuminated based on the result of processing data from monitoring a fluid path. For example, in one embodiment a red LED light may illuminate on the PPG sensor to indicate that a specific fluid path is not meeting pre-defined performance characteristics such as flow or carry-over. This would allow a service technician to easily identify which fluidic device and what portion of a fluidic device is not meeting performance standards. The PPG sensor may also have a built in power source, such as a battery.

The PPG device may also be associated with a RFID device where the RFID device can be used to ‘turn on’ the PPG sensor based on the RFID device being activated by a RFID reader module associated with an interface device or the diagnostic platform. When the RFID device is energized by an RFID reader module, the RFID device circuitry may be connected to circuitry associated with the PPG sensor (such as a relay, reed switch, thermal switch/sensor, or inductor) which may be configured to detect an output of the RFID device when it is energized (such as a milliamp output or magnetic field). The connection between the RFID device and the PPG sensor may be a physical connection and/or may be performed by a non-contact method where the two devices are in close proximity but are not physically in contact. The RFID device circuitry may also be fully integrated into the PPG sensor circuitry. The use of a RFID device (or RFID device sensor) can allow for ‘on demand’ actuation and control of the PPG sensor which can be used to extend the life of the sensor by limiting when the battery is used by the sensor.

The PPG sensor may also have capabilities to modify the ‘detection zone’ associated with the PPG sensor by modulating the power associated with the light source that is used for detection. For example, the PPG sensor may have the ability to ‘auto tune’ its light source output with respect to the size of the fluid path or feature (such as a fluid well/chamber) it is monitoring by performing its own diagnostic routine which it would use to verify light absorption associated with a fluid material that could be used to calibrate the fluid path for use with the PPG sensor, such as deionized water or a sheath fluid. By measuring the fluid path with a known substance, the PPG sensor could determine its optimal output configuration based on the size of the specific fluid path. The PPG sensor may also receive instructions for how it should perform from either the interface device software and/or the diagnostic system software.

The PPG sensor may also use software algorithms to filter out signal noise that may be generated during the detection process. For example, signal noise from other light sources in the diagnostic platform or reflection of light in the fluid device substrate may be detected and filtered out to allow the PPG sensor the ability to extract the target signals associated with the flow of fluid in a fluid path. The software algorithms may be able to detect what a target fluid path signal looks like and ignore other income signals that are generated from sources that are not part of the fluid path. The fluidic device may also use shielding to better isolate outside signal noise to allow the PPG sensor to improve its performance. The PPG sensor may also be able to detect pulsations of the fluid path which may be the result of a device that is responsible for fluid movement, such as a pump.

It should be appreciated that pressure pulsations are critical in certain applications, and the PPG sensor may be able to detect the peaks and valleys (and/or the space/interval and/or frequency between pressure pulse peaks, peak-to-peak measurement) of a pulse wave using light absorption/transmission/refraction methods. This may allow the PPG sensor to output a signal that can also qualify the fluid path pulsation which can be used to monitor the performance of a pump that is upstream from the PPG sensor and can also be used as a quality metric for the fluid path performance since the pulsation of a fluid path may have a threshold that is critical to a test or process (such as a mixing process) that is performed on the fluid (such an optical detection of blood cells using a laser light source).

In one embodiment, the PPG sensor may also be able to detect the fluidic device material transmission properties as a way to select an appropriate detection configuration set up, or even decide which light source to use since a PPG sensor may contain more than one light source type allowing for the ability to apply the most appropriate light source solution for the fluidic device substrate. For example, if a PPG sensor detects (or is told by a mobile device, an interface device, and/or the diagnostic platform) that it is on a fluidic device substrate made of Acrylic, it may select a green LED light source for detection of fluid since the 540 nanometer wavelength range would be acceptable for the detection environment. If the PPG sensor were to detect (or is told by a mobile device, an interface device, and/or the diagnostic platform) that it is on a fluidic device substrate made of ULTEM 1000 material, it may select an infrared light source since the wavelength range of 700 nanometers to 1000 nanometers may be more suitable for the detection environment.

The invention also includes the use of the interface device (‘SmartHub’) to detect physical characteristics associated with a fluid bag (such as an intravenous bag). It is contemplated that this fluid bag may be located in any location, such as a hospital room or a person's home. The fluid bag may have at least one RFID device sensor attached to it (or that is printed onto the fluid bag/intravenous bag) that is able to detect at least one physical characteristic and/or fluid type associated with the bag, such as the amount of fluid in the bag, the type of fluid in the bag, the temperature of the fluid in the bag, and the pressure associated with the fluid in the bag related to the area that the RFID device sensor is able to measure. The bag may also be part of an assembly which has components connected to it such as a ‘drip chamber’ which may be used to allow a gas (such as air) to rise out from fluid so that it is not passed downstream to the patient. The fluid bag may have at least one RFID device sensor to detect fluid level, and a component connected to the fluid bag (for example, drip chamber) may also have at least one RFID device sensor to detect fluid level. By using multiple RFID device sensors, the interface hub software would be able to detect and confirm that fluid is flowing between the fluid bag and the drip chamber by monitoring the outputs of both RFID device sensors.

For example, a sodium chloride intravenous bag would have a RFID device sensor near the bottom of the bag close to the fluid ports, where the RFID device sensor would be activated by the interface device and report back a value or data set associated with the fluid bag's fluid level. The drip chamber would also have an RFID device sensor and would be activated by the interface device and report back a value or data set associated with the drip chamber's fluid level. If there was an obstruction at the outlet port of the fluid bag due to debris, the drip chamber fluid level may be affected by this. The interface device would be configured to monitor the fluid status of both of the fluid bag and the drip chamber to make sure fluid levels were consistent and there were not issues with blockage that could cause issues with the patient receiving the fluid. Instead of having the nurse in a hospital have to walk around to the hospital rooms to ensure that the intravenous bags in patient rooms are operating correctly, the interface device would be able to report a status (real-time and/or delayed as desired) to a nurse computer station (or a mobile device the nurse has on them) allowing the nurse to receive real-time updates and alerts based on fluid levels.

The RFID device sensors that are associated with the fluid bag and the drip chamber may be detected by the interface device so that they are associated with a patient (or a fluid bag assembly station). The software on the interface device would be able to be configured by a nurse (either remotely through a nurse computer station or a mobile device) to allow for the room number, floor number, patient ID number to be associated with the received data from the RFID device sensors from a fluid bag assembly. The interface device may generate data that is sent to the hospital network/nurse computer station software such as continuous status of fluid levels in a fluid bag assembly or generate emergency status codes based on information collected by the RFID device sensor(s). The interface device may also be able to communicate with devices that are worn by a patient, such as a RFID device or a Bluetooth enabled patient tag where the interface device is able to receive patient ID information from the device associated with a patient(s) which can be used to identify the data that was received by the sensors on the fluid bag assembly which can also be sent by the interface device to the computer system in a hospital.

It should be appreciated that the functionality of the interface device may also exist on the patient tag device and a mobile device used by the hospital staff which would have circuitry and software capabilities to both communicate with the RFID device sensor(s) and the may also be able to communicate with at least one of the hospital network system and/or a mobile device used by the hospital staff. The interface device teaching/learning capability would allow a nurse to set up and configure the software of the interface device to associate at least one fluid bag RFID device sensor and/or at least one drip chamber RFID device sensor with each other (to create a matched pair) using a mobile device software interface or a portable computer interface, where the software on either mobile device or portable computer would allow the nurse to identify the RFID device sensors that will send/communicate data from a specific fluid bag assembly to the interface device.

The interface device software may be configured to store this set up configuration and identify unique identification information from the RFID device sensors, such as a EPC code or other unique identifier found in the user memory of the RFID device sensor, in order to know how to match the received data to a specific patient and/or fluid bag assembly station. The functionality of the interface device may also exist as part of a display screen (and/or TV screen) or objects such as a light fixture, or computer system that is present in the hospital room (or patient's home). The interface device would also be able to capture data from the RFID device sensor tag which may relate to the fluid type that is inside of the fluid bag, the expiration date of the fluid bag as well as recommended temperature for storage. For example, a hospital or a blood bank facility with a supply of blood transfusion bags stores whole blood at 1.0° C.-6.0° C. for 35-45 days and may also store platelet concentrate which may be stored for only 5-7 days at room temperature. The interface device is able to monitor the data from the RFID sensors and compare the data against pre-determined thresholds set up specifically for the fluid bag the system is monitoring (for example, the characteristics and storage requirements of blood bags).

The interface device would be able to detect RFID device sensors which are able to measure the temperature of the environment associated with the blood bag (and/or the temperature of the fluid bag itself) and would be able to monitor the amount of time a blood bag has been stored and at what temperature. The interface device would be able to communicate the total number of days a blood bag has left before it is no good, where this data could be written/programmed back to the RFID device sensor memory bank (or another RFID device that also has available memory) which would allow a nurse or technician to be able to quickly identify a blood bag's useful life by scanning the RFID device sensor with a mobile device/RFID reader or by using a computer system which is in communication with the interface device, where the interface device has already posted the hourly/daily/weekly information about stored blood bags to either local or remote computer system (such as a hospital network or a blood bank facility computer network).

The interface device may include software and/or hardware which is/are configured to determine how a blood bag RFID device sensor tag status should be determined. For example, the interface device software may have pre-set threshold values stored in its memory specific with a certain type of fluid bag which will allow the interface device to determine how to identify the status (good or not good) of the fluid bag. The status of the fluid bag can be programmed to the RFID device sensor memory bank by the interface device (or a mobile device). A nurse (or other employee) who may be wearing a device with a heads-up-display (HUD, such as Google Glass and/or an Apple® Watch) can walk into a hospital room and have a visual status of the fluid bag assembly levels as displayed to the nurse on their HUD or display of a mobile device.

The interface of the HUD device (mobile device) may also allow a nurse to provide feedback to the hospital system, such as they have confirmed that the fluid bag operation is acceptable. For example, a nurse may walk into a hospital room and information about the fluid bags may appear on her HUD screen. The nurse may then use the touch navigation sensor on the Google Glass device or touch screen on their Apple® Watch to navigate to a menu that allows her to submit a confirmation that she was in the room and that everything was OK. This information can be send to the hospital network where it can be logged and timestamped as part of a daily log report that is used for quality and safety control by the hospital.

Still yet another embodiment of the invention includes a hospital bed (or other article, such as a chair, gurney, etc.) which has the ability provide power so an interface device would be able to be part of a hospitable bed. A hospital bed may also have electronics that could also have the same capability of an interface device. The interface device may also have its own power supply source in the event there is no device it can connect to that can provide power.

Still yet another embodiment includes the use of an interface device as part of a system that is used for the delivery of chemotherapy fluids as part of a cancer treatment program. An infusion pump may have the capabilities of the interface device to establish communication with RFID device sensors that are associated with fluid bags/fluid bag assemblies that are used by the infusion pump control system. This would allow the infusion pump control system to benefit from data that the RFID device sensor can generate (fluid type information, fluid bag temperature information, fluid bag expiration information, fluid bag level/fluid bag assembly level information, etc.). For example, if a nurse was setting up an infusion pump machine as part of a cancer treatment session, the infusion pump control system would be able to detect information such as expiration data for a fluid bag, or first look to see if the fluid bag RFID device sensor had been identified as ‘do not use’ previously as part of a hospital inventory management system.

If the infusion pump control system detects a fluid bag product that is expired or has a RFID device sensor tag that is identifying itself as ‘do not use’, the infusion pump control system software will not run and will prompt the nurse to replace the fluid bag with another fluid bag. During the use of the system during a treatment session, the infusion pump control unit may gather data from the RFID device sensors continuously and/or semi-continuously so the software of the infusion pump control unit is able to detect that status of fluid levels for the respective fluid bags that are used as part of the treat process. This may also include fluid bags that are not directly influenced by the infusion pump control system but may be used by the patient as part of the cancer treatment process such as fluid bags to improve hydration of the patient. If the infusion pump control unit were to identify a fluid level issue with any fluid bag, the software of the infusion pump may decide to pause or stop operation and alert the user of the infusion pump control unit and direct them to troubleshoot the specific fluid bag assembly. The infusion pump control unit may also have a communication port that allows an external interface device to provide the above capabilities and to also communicate information to the infusion pump control unit.

It is also contemplated that the infusion pump control system may also be configured to identify the type of fluid contained in the fluid bag and the type of fluid that is intended to be infused into a patient and if the fluids don't match, then the software of the infusion pump may pause or stop operation and alert the user of the infusion pump control unit that the wrong fluid is about to be used.

Still yet another embodiment which benefits from the interface device capabilities is a cooler that is used to transport critical fluids, such as blood bags. The cooler may have the interface device as part of the cooler assembly or it may be added as an aftermarket addition to the cooler. The cooler may have sensors installed as part of the cooler assembly that detect if the cooler lid is shut entirely. For example, there may be a magnetic reed switch signal that is detected by the interface device software, where the software will monitor that status of the reed switches. As described earlier, the learning capability of the interface device will allow a person who is responsible for loading up the portable cooler unit to identify the blood bag as a good bag (since the RFID device sensor that is on the blood bag may have a status indicator), and if the blood bag is identified as good, the person will proceed with placing the blood bag in the cooler.

The interface device may benefit from the display screen described earlier, allowing for a real time identification of a status indicator for the blood bag(s) to be displayed on the display screen which shows how many blood bags, temperature of each blood bag, and how many days each blood bag has left before it expires. If the interface device is at least partially external to the cooler unit, this would allow a person to see the display screen of the interface device and get a real time status of the contents of the cooler (blood bags for example) without having the need to open the cooler door, since the RFID reader module that is part of the interface device would be able to communicate with the RFID device sensor(s) inside of the cooler. The interface device will also monitor and collect data from another RFID sensor which is placed inside of the cooler to monitor the ambient cooler environment. The ice packs inside of the cooler may also have RFID device sensors to allow the interface device to collect data on the temperature of the ice packs as well.

The interface device can have its own built in power supply, or it may use a power supply built into the cooler unit. If the interface device does not detect any fluid bags with RFID devices, the interface device can go into power management mode and wake up periodically to check if there are any fluid bags present. Additionally, when the cooler door is opened and the switches are in their open status, this may also be used to trigger the interface device software to start scanning for RFID device sensor tags associated with fluid bags. If the cooler were to detect a temperature of a blood bag that went beyond the safe temperature range of the fluid bag, the interface device can write a ‘kill command’ to the blood bag RFID device sensor which will permanently make that blood bag identified as no good. The interface device can also identify what the failure mode was for the blood bag (example: over exposure to temperature, expired date of the fluid bag) and write this data to the RFID device sensor of the fluid bag as well.

The invention may benefit from the use of communication protocols such as EPC Gen2, NFC protocols, etc, Bluetooth, Zigbee, Wifi, Infrared, 800-900 mHz communication methods, and any combination of protocols to produce the desired end result. It should be appreciated that a diagnostic platform can include (but is not limited to) systems such a hematology system, urine analysis system, DNA sequencing system, chemotherapy management system, clinical chemistry system, water analysis system, immunoassay system, titrator system, blood glucose testing system, flow cytometry system, cell sorting system, drug discovery system, processes used in manufacturing for medicine discovery such as mixing manifolds.

Level sensor assemblies such as float switch assemblies (single point or multi-point float switches) are prone to issues with fluids that are in a reservoir (for example, such as a sheath fluid which may have foam and result in crystallization of salt particles causing a float to ‘stick’ in a unwanted position) which can cause particulate to dry when the fluid level is reduced. This can cause the float switch sensor to stick or even possibly interrupt a reading of the embedded magnet (or magnetic sensor) that is disposed within the float switch assembly.

While the invention disclosed herein describes wireless level sensing technologies which can be used on the outside of a fluidic device or fluid reservoir, there is also an opportunity to improve how other level sensor solutions can be delivered to the market. For example, Diba Industries offers for sale a continuous level sensor solution called ‘Hydroplus’ (https://www.dibaind.com/diba-technologies/hydroplus/) which uses a pressure transducer electronic chip assembly to detect the constant changes in volumetric pressure that is present within a column that is at least partially submerged into a fluid reservoir. Unlike the float switch designs that are typically used and have failures directly associated with components that move when the fluid level changes in a fluid reservoir, this approach offers a ‘no moving parts’ design eliminating the failures associated with float switches while also offering opportunities for improved material compatibility characteristics. The output of the pressure transducer may be a voltage or current type output signal that is made available to a machine such as a diagnostic platform.

The limitation with the above mentioned continuous level sensor (or semi-continuous) solution is that existing platforms that use float switch assemblies are not easily retrofitted with a ‘no moving parts’ solution of the electronic transducer level sensing solution because the output signal is not typically equal to that of a float switch. Existing diagnostic platforms may often times fall under FDA approvals and the farther a replacement option is from a current implemented design the harder it will be to justify engineering resources to change a design, even if the solution is better.

Still yet another invention of this application is aimed at solving that problem. A level sensor assembly can be designed to have added intelligence which would allow for a precise control of the output signal (or data) so as to match a diagnostics machine input requirement. For example, a circuit board containing at least one pressure transducer electronic chip could be interfaced directly with a microprocessor. The output of the pressure transducer chip (voltage or current output) would be directed into the microprocessor. The microprocessor may contain interface ports that would allow for a direct communication link with a pressure transducer, as well as communication port(s) for output signal delivery to a diagnostic platform, and communication port(s) that would allow for the microprocessor to be programmed using a separate device (such as a computer or a mobile device).

A mobile device may be able to communicate (either wired or wirelessly) with the assembly allowing for a portable user interface option that would let a service technician apply either a pre-set level sense operation program into the assembly, or would also allow for the ability to teach/define a ‘zone’ for a pre-determined level sense output. For example, a mobile device might have an interface that would let the technician assign a first level sense output based on a detected fluid level using the input of the pressure transducer. The technician would be able to assign a name, output type (command response, voltage output, current output, etc.) for this defined fluid level which would be updated into the microprocessor software. This process could be repeated multiple times for different fluid level positions until the desired amount of fluid level positions is captured.

As described herein, a pre-set fluid level program could also exist on the mobile device, and/or the mobile device may be able to communicate with a remote web server and retrieve a list of approved level sensing programs for a particular fluid reservoir size. The mobile device may be able to scan a barcode/QR code attached to the level sensor assembly or the fluid reservoir which may contain identifying information within the barcode/QR code to be used to identify the specific fluid level sensor program that may be programmed into the PCB level sensor assembly microprocessor. RFID tags/NFC tags can also be used for allowing the mobile device to identify what specific program should be used for a specific fluid level sensor assembly. Once the mobile device determines which program should be used to program the microprocessor, a communication link may be automatically established with at least one microprocessor associated with a level sensor assembly in order to program the software.

It should be appreciated that the software residing in the microprocessor may be programmed to receive, process, and identify a pre-determined input level of the pressure transducer chip (i.e. a voltage level or current level) where the pre-determined input level is compared with an internally stored input threshold value that is stored in memory within the microprocessor (or memory associated with the microprocessor to include external memory chips). The software may compare the input level signal (or semi-continuously compare) against the internally stored pre-determined input signal threshold value and once the pre-determined threshold value is met (or exceeded) by the input signal, the software may generate a resultant output signal (or data command response value) which can be made available to the diagnostic machine. The software may also generate a command response value based on the pre-determined threshold value being met by the input signal, where the command response value may also be stored in memory of the microprocessor allowing the diagnostic machine software to request a status command response on liquid level status periodically from the level sensor electronics. By using a command based response scheme, this would eliminate any noise issues associated with outputting a data signal value to a diagnostic machine since the command value would be easily interpreted by the diagnostic machine software and would not require any conversion from an analog to digital signal.

This invention would advantageously allow for a single pressure transducer chip working with a microprocessor (as described above) to provide both single point and multi-point level sensing capabilities just like a common float switch assembly, but without the problems associated with a float switch assembly. The software residing in the microprocessor may be able to store multiple pre-determined threshold signal values where each value is associated with a corresponding input signal value that would come from the pressure transducer chip.

The output signal generated by the pressure transducer chip and the corresponding pre-determined threshold value (or value range) residing inside of the software could be correlated to the physical position of a float switch that was previously used by the diagnostic machine, or can be determined by a physical volume level of the fluid container. For example, a multi-point level sensing solution requiring four different level values could generate four unique output signals (or command responses) related to four different fluid level values such as: HIGH, HALF, LOW, VERY LOW, etc. Part of the output of the microprocessor/pressure transducer assembly may also include information related to the calibration date of the pressure transducer chip. This would allow the diagnostic machine software application to detect upcoming calibration and/or overdue calibration requirements which could result in automatic service alerts as described earlier in the above application. The number of times a pressure transducer has been actuated may also be used as part of an output by the system so the diagnostic machine would have the ability to know how much useful life is left on the pressure transducer component.

The software residing in the microprocessor assembly may also control when the pressure transducer chip is powered. This could prolong the useful life of the pressure transducer chip component by drastically reducing the amount of the time the pressure transducer chip needs to be actuated. Since most level sensing applications require ‘on-demand’ status, the software may be configured to power the transducer when the diagnostic machine software first communicates with the microprocessor software (and/or based on pre-determined timing intervals that are set up within the microprocessor software). An on-board battery (power supply) may be used to provide power to the assembly, or external power may be provided by the diagnostic machine hardware.

The microprocessor may also benefit from working with external circuitry components that may be needed to generate a desired output signal to the diagnostic machine. The pressure transducer and microprocessor and all components requirement to deliver the desired end output signal/command may also be fully integrated as part of a single PCB assembly or be partially integrated as part of at least two assemblies that are in signal communication with each other (PCB board assembly mother board and daughter board). The microprocessor may also contain all of the pressure sensing and output signal generation capabilities.

The PCB assembly may also contain noise filtering circuitry (high pass, low pass, etc.) which can be used to isolate the output signal of the pressure transducer (and/or the microprocessor) based on vibration and/or electrical noise sources. The noise filtering circuitry may be implemented in a way where multiple noise filtration circuits can exist on the assembly and where there is a noise/signal monitoring circuit on the output of the pressure transducer which is able to detect the presence of signal noise and then apply the correct noise filtering circuit based on the analysis of the noise in order to keep the output signal clean. This noise monitoring capability may also be part of the microprocessor capabilities. Multiple noise filtering circuits may be used together as part of the microprocessor software logic in order to optimize the output of signal quality in real time based on the environmental conditions for where the diagnostics machine is installed which may also contribute to unknown noise sources.

In still yet another embodiment, the assembly may also be integrated as part of a bottle cap assembly design (or also be external) which would allow for easy integration into existing fluid reservoir designs.

As described above, the methods and embodiments described hereinabove and in the several figures may be embodied in the form of computer-implemented processes and apparatuses for practicing those processes. The methods and embodiments described hereinabove and in the several figures may also be embodied in the form of computer program code containing instructions embodied in tangible media, such as floppy diskettes, CD-ROMs, hard drives, or any other computer-readable storage medium, wherein, when the computer program code is loaded into and executed by a processor, the processor becomes an apparatus for practicing the invention. Existing systems having reprogrammable storage (e.g., flash memory) may be updated to implement the invention. The methods and embodiments described hereinabove and in the several figures may also be embodied in the form of computer program code, for example, whether stored in a storage medium, loaded into and/or executed by a computer, or transmitted over some transmission medium, such as over electrical wiring or cabling, through fiber optics, or via electromagnetic radiation, wherein, when the computer program code is loaded into and executed by a computer, the computer becomes an apparatus for practicing the invention. When implemented on a general-purpose microprocessor, the computer program code segments may configure the microprocessor to create specific logic circuits. It should be further appreciated that the methods and embodiments described hereinabove may also be practiced, in whole or in part, via any device suitable to the desired end purpose, such as a computer, iPod, MP3 Player, smartwatch, tablet, wearable device with heads up display capability, a PDA, a Pocket PC and/or a Cell phone with connection capability.

While the invention has been described with reference to an exemplary embodiment, it should be understood by those skilled in the art that various changes may be made, and equivalents may be substituted for elements thereof without departing from the scope of the invention. Moreover, the embodiments or parts of the embodiments may be combined in whole or in part without departing from the scope of the invention. In addition, many modifications may be made to adapt a particular situation or material to the teachings of the invention without departing from the scope thereof. Therefore, it is intended that the invention not be limited to the particular embodiment disclosed as the best mode contemplated for carrying out this invention, but that the invention will include all embodiments falling within the scope of the appended claims. Moreover, unless specifically stated any use of the terms first, second, etc. do not denote any order or importance, but rather the terms first, second, etc. are used to distinguish one element from another.

I claim:

1. A fluid monitoring system, comprising:
an RFID sensing device, wherein the RFID sensing device is configured for wireless communication;
a fluid reservoir, wherein the RFID sensing device is associated with the fluid reservoir;
an interface device, wherein the interface device is configured to establish a communication link with the RFID sensing device and a remote processing device, and wherein the interface device includes,
an RFID processing device configured to process RFID sensor data received by the RFID sensing device to generate a processed RFID result; and
a remote processing system, wherein the remote processing system is configured to,
receive the processed RFID result from the RFID processing device,
process the processed RFID result to generate output data, and
operate responsive to the output data.

2. The fluid monitoring system of claim 1, wherein the fluid reservoir contains at least one of a diagnostic system reagent fluid, biological sample of a person or animal, a waste fluid, an intravenous fluid, blood, urine, saline solution, diagnostic system wash fluid, patient medicine, patient drug treatment fluid

3. The fluid monitoring system of claim 1, wherein the RFID sensing device is at least one of a temperature sensor, a fluid sensor, a moisture sensor, a conductivity sensor, a pressure sensor, a light sensor

4. The fluid monitoring system of claim 1, wherein the RFID sensing device is at least one of a low frequency sensor, an ultra-high frequency sensor, and a near field communication sensor

5. The fluid monitoring system of claim 1, wherein the wireless communication link with the RFID sensing device is a wireless bi-directional communication link, and
wherein the interface device is further configured to send information to a memory location associated with the RFID sensing device.

6. The fluid monitoring system of claim 1, wherein the communication link with the remote processing system is at least one of a hardwired or wireless connection, and
wherein the interface device is further configured to receive information from a remote computer system and operate in response to the received information.

7. The fluid monitoring system of claim 1, wherein the interface device includes interface device memory and processes the RFID sensor data by at least one of,
comparing the RFID sensor data with a pre-determined value that is stored in a memory location associated with the interface device and generating an output based on this comparison, and
generating an output response that combines the processed RFID sensor data with information that is stored within the interface device memory.

8. The fluid monitoring system of claim 1, wherein the remote processing system is at least one of a diagnostic machine control system, a mobile device, a hospital patient management system, an infusion pump system.

9. The fluid monitoring system of claim 1, wherein the output data is used to by the remote processing system to control at least one of a display associated with the computer system, a status notification that can be generated by the remote processing system, and generating a communication that can be sent by the computer system to the interface device.

10. The fluid monitoring system of claim 1, wherein in the interface device is at least partially integrated with the remote processing system

11. A method for monitoring a fluid reservoir, the method comprising;
configuring an interface device having a processor to establish a first communication link with an RFID sensing device associated with a fluid reservoir;
processing data received from the RFID sensing device via the interface device to generate output data;
configuring the interface device to establish a second communication link with a remote processing system;
receiving the output data from the interface device;
processing the output data via the remote processing system to generate resultant data; and
operating the remote processing system responsive to the resultant data.

12. The method of claim 11, wherein the communication link between the RFID sensing device and the interface device is wireless.

13. The method of claim 11, wherein processing the data received from the RFID sensing device includes at least one of comparing the data received from the RFID sensing device with a pre-determined value that is stored in memory associated with the interface device and generating an output based on this comparison, and generating an output response that combines the processed RFID sensor input data with information that is stored inside of the interface device memory.

14. The method of claim 11, wherein the RFID sensing device is at least one of a temperature sensor, a fluid sensor, a moisture sensor, a conductivity sensor, a pressure sensor and a light sensor.

15. The method of claim 11, wherein the fluid reservoir contains at least one of a diagnostic system reagent fluid, a biological sample of a person or animal, a waste fluid, an intravenous fluid, blood, urine, saline solution, a diagnostic system wash fluid, patient medicine, patient drug treatment fluid.

16. The method of claim 11, wherein the RFID sensing device is at least one of a low frequency sensor, an ultra-high frequency sensor, and a near field communication sensor.

17. The method of claim 11, wherein the remote processing system is at least one of a diagnostic machine control system, a mobile device, a hospital patient management system, an infusion pump system.

18. The method of claim 11, wherein the first communication link is a wireless, bi-directional communication link and wherein the interface device is further configured to send information to a memory location associated with the RFID sensing device.

19. The method of claim 11, wherein the resultant data is used to by the remote processing system to control at least one of a display associated with the remote processing system, a status notification that can be generated by the remote processing system, and generating a communication that can be sent by the remote processing system to the interface device.

20. An interface device for monitoring at least one characteristic of a fluid reservoir, the interface device comprising:
circuitry configured to establish a wireless communication link with an RFID sensor;
at least one processor configured to process data that is received by the RFID sensor and generate output data;
circuitry configured to establish a communication link with at least one computer system;
and
a display device associated with the interface device, wherein the display screen provides a user interface and access to the interface device functionality.
